SEQUENCE LISTING (8 sequences)

SEQ ID NO: 1 - DNA, 250 bp, artificial sequence
  genomic DNA sequence portion of proximal 5′ region of p11Beta gene
ctaaataaagtttggagaaaaatcacctgtcaagtgctggttaactaaatttcttaggaccctgtaatccttaagcatgcagtttattcagatgtcaaatatttatgttgaatttgaatcaatgtggtgtgtgtgtgtgtacatatgtatgtgtatatgtttttatttatatgtatataagaataatttttttcctttagttggtggtatggattggataatgtacatatatataatttgtgaagtgata

SEQ ID NO: 2 - DNA, 3424 bp, artificial sequence
  cDNA sequence encoding p110Beta polypeptide; Genbank Acc. No. Z29090
aggatcagaacaatgcctccaagaccatcatcaggtgaactgtggggcatccacttgatgcccccaagaatcctagtggaatgtttactaccaaatggaatgatagtgactttagaatgcctccgtgaggctacattagtaactataaagcatgaactatttaaagaagcaagaaaataccctctccatcaacttcttcaagatgaatcttcttacattttcgtaagtgttacccaagaagcagaaagggaagaattttttgatgaaacaagacgactttgtgatcttcggctttttcaaccatttttaaaagtaattgaaccagtaggcaaccgtgaagaaaagatcctcaatcgagaaattggttttgctatcggcatgccagtgtgcgaatttgatatggttaaagatcctgaagtacaggacttccgaagaaatattcttaatgtttgtaaagaagctgtggatcttagggatcttaattcacctcatagtagagcaatgtatgtctatccgccacatgtagaatcttcaccagagctgccaaagcacatatataataaattggatagaggccaaataatagtggtgatttgggtaatagtttctccaaataatgacaagcagaagtatactctgaaaatcaaccatgactgtgtgccagaacaagtaattgctgaagcaatcaggaaaaaaactagaagtatgttgctatcatctgaacaattaaaactctgtgttttagaatatcagggcaagtacattttaaaagtgtgtggatgtgatgaatacttcctagaaaaatatcctctgagtcagtataagtatataagaagctgtataatgcttgggaggatgcccaatttgaagatgatggctaaagaaagcctttattctcaactgccaatggactgttttacaatgccatcttattccagacgcatttccacagctacaccatatatgaatggagaaacatctacaaaatccctttgggttataaatagagcactcagaataaaaattctttgtgcaacctacgtgaatctaaatattcgagacattgacaagatttatgttcgaacaggtatctaccatggaggagaacccttatgtgacaatgtgaacactcaaagagtaccttgttccaatcccaggtggaatgaatggctgaattatgatatatacattcctgatcttcctcgtgctgctcgactttgcctttccatttgctctgttaaaggccgaaagggtgctaaagaggaacactgtccattggcatggggaaatataaacttgtttgattacacagacactctagtatctggaaaaatggctttgaatctttggccagtacctcatggattagaagatttgctgaaccctattggtgttactggatcaaatccaaataaagaaactccatgcttagagttggagtttgactggttcagcagtgtggtaaagttcccagatatgtcagtgattgaagagcatgccaattggtctgtatcccgagaagcaggatttagctattcccacgcaggactgagtaacagactagctagagacaatgaattaagggaaaatgacaaagaacagctcaaagcaatttctacacgagatcctctctctgaaatcactgagcaggagaaagattttctatggagtcacagacactattgtgtaactatccccgaaattctacccaaattgcttctgtctgttaaatggaattctagagatgaagtagcccagatgtattgcttggtaaaagattggcctccaatcaaacctgaacaggctatggaacttctggactgtaattacccagatcctatggttcgaggttttgctgttcggtgcttggaaaaatatttaacagatgacaaactttctcagtatttaattcagctagtacaggtcctaaaatatgaacaatatttggataacttgcttgtgagatttttactgaagaaagcattgactaatcaaaggattgggcactttttcttttggcatttaaaatctgagatgcacaataaaacagttagccagaggtttggcctgcttttggagtcctattgtcgtgcatgtgggatgtatttgaagcacctgaataggcaagtcgaggcaatggaaaagctcattaacttaactgacattctcaaacaggagaggaaggatgaaacacaaaaggtacagatgaagtttttagttgagcaaatgaggcgaccagatttcatggatgccctacagggcttgctgtctcctctaaaccctgctcatcaactaggaaacctcaggcttaaagagtgtcgaattatgtcttctgcaaaaaggccactgtggttgaattgggagaacccagacatcatgtcagagttactgtttcagaacaatgagatcatctttaaaaatggggatgatttacggcaagatatgctaacacttcaaattattcgtattatggaaaatatctggcaaaatcaaggtcttgatcttcgaatgttaccttatggttgtctgtcaatcggtgactgtgtgggacttattgaggtggtgcgaaattctcacactattatgcaaattcagtgcaaaggcggcttgaaaggtgcactgcagttcaacagccacacactacatcagtggctcaaagacaagaacaaaggagaaatatatgatgcagccattgacctgtttacacgttcatgtgctggatactgtgtagctaccttcattttgggaattggagatcgtcacaatagtaacatcatggtgaaagacgatggacaactgtttcatatagattttggacactttttggatcacaagaagaaaaaatttggttataaacgagaacgtgtgccatttgttttgacacaggatttcttaatagtgattagtaaaggagcccaagaatgcacaaagacaagagaatttgagaggtttcaggagatgtgttacaaggcttatctagctattcgacagcatgccaatctcttcataaatcttttctcaatgatgcttggctctggaatgccagaactacaatcttttgatgacattgcatacattcgaaagaccctagccttagataaaactgagcaagaggctttggagtatttcatgaaacaaatgaatgatgcacatcatggtggctggacaacaaaaatggattggatcttccacacaattaaacagcatgcattgaactgaaagataactgagaaaatgaaagctcactctggattccacactgcactgttaataactctcagcaggcaaagaccgattgcataggaattgcacaatccatgaacagcattagatttacagcaagaacagaaataaaatactatataatttaaataatgtaaacgcaaacagggtttgatagcacttaaactagttcatttcaaaa

SEQ ID NO: 3 - protein, 1068 aa, artificial sequence
  p110Beta polypeptide, Genbank Ac. No. Z29090
MPPRPSSGELWGIHLMPPRILVECLLPNGMIVTLECLREATLVTIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPHVESSPELPKHIYNKLDRGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLKMMAKESLYSQLPMDCFTMPSYSRRISTATPYMNGETSTKSLWVINRALRIKILCATYVNLNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQERKDETQKVQMKFLVEQMRRPDFMDALQGLLSPLNPAHQLGNLRLKECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

SEQ ID NO: 4 - DNA, 8890 bp, artificial sequence
  encodes p110Beta polypeptide; Genbank Acc. No. AJ297549
ttaaccctcactaaagggagtcgactcgatcttcaaaggctgctctgaagtgatttgcttaattttaggactagtgccttaataacatgtgtagtgactgtgtagttttgttcgtgtttatggaagtttattggttaagagttcctgagtatagagacctttccatatactgtggtgagaaacagtactttaaaaagcatgttgacaatacctgaattctgtgatcaatcttagcatggctaaaagtaagaccactagacattatatctcctcaaggagcttataatgaatccatatttaaatttcccagattatttcaaagataggttttgtttgtttgttttttgttttttttgagatggatttttgctcttgttgcccaggctggagtgcaatggcacaatctctgctcactgcaacctccacctcttgggttcaggcgattctcctgcctcagcctcctgagtagctgggattacaggcttgaaccaccatgcctggctaatttttttgtatttttagtagagacagggtttcgccatgtcagtcaggcttgtctcaaactcctgacctcaggtgatccacccacctcggcctcccaaagtactgggattacaggcataagccaccgtgcctggcccaaagatagttggttttttttttttgagacagagtcccactctgttgcccaggctggagtgcagtggcgtgatctcagctaactgcaacctccaccttcctggttcaagtgattcttctgcctcagcctcccgagtagctgggactacagacacacgccaccacccccgactaatttttgtatttttatttttttgagacgaagtctcgctgtgttgcccaggctggagtgcaatggtgctgtctaggctcactgcaacctctgcctcccaggttcaagcaattctcgtgcctcagccttctgagtagctgggattacaggcacccgccaccatgcctggctaatttttgtatttttagtagacatggggtttcaccatattggccaggctggcctcgaactcctgaccttgtgatccacctgccttggcctcccaaagtgctgagattacaggcatgggccacagtgcctggctaagatagttttttatagttgatttgttccagctaacattggatttggctgtgtctcctaagtgttttttaaattctttaacagtccttccaccatcttcttcctcctcttcttgatattgattagctggagaaaccatataatttgttttattgaagatgccacattctctacttggctgattgcttccttgaagtatttaacttttttctctgtttcctgtaaactagcacatagatcagatctaaagccttgattagattcaagttcagttttgttttgtgtgaatggtaagtactgactattgcatttcatgaggagacacataatgtctgatagtcccacttttagtaatgctaagatcaggggattcctgtagtgtcaacaagacagacacggacgattgtaaagtttcttctcctttcaccttattattatttatgtctttagcatctattaatgatcattgcctagatctgttagttcattagcacttgcagaattgtgattttctaatttattattctttttccatttattagcaaaagaagttctaaaaattgttttttaatttttatttttttaagacagggtctcactttgttgtccaggctggagtgcagtggtactattttagctcaatgcagccttagattcttggactcaagcaatcctcatgcctcagtctcccaagtagctgggactacaggtatgcaccactacgcctggccaattttttgtatgttttgtagagacagggtcttgtttgttgcccaggctggtctcaaactcctggactcaagcaatccacccatctaagcctccccaagtaatggtattacaggcatgggccactgtgcccggcctcctgtctcttttgatatgacactttcattaatggcataattgttaaatgaaccttggttcatttaactttagcgctcatgttcttccaattattattgttgtatcttcttgaaggaagcccttaaggaataggtaagacttaaatggcactattaaagggtgcatttgttgggatagtgcgtttgttttgatatgaggagtttattcctcatgtgaacataggtctagcagcaggaaggcagaaggcactgtgataccagtctcgggctgtagagtgatgggagagcaagaaaaataggccagagaaatagcacaaggggccttgccatctgtattgagcgatttggtgtttttaatgcttaggggatgggccgtgaggttgatagaaaattgtccttgttggctgggcacggtgggtcacgcctgtaatcccagcactttgggaggccaaggcgggcggatcacgaggtcaggagatcgagaccatcctggctaacacggttgaaaccccgtctctactaaaaatacaaaaaatttagccaggcatggtggcaggcgcctgtagtctcagctactcgggaggctgaggcaggagaatggcgtaaaactgggaggcggagcttgcagtgagctgagatgtcactactgcattccagcctaggcaacagagagagactccatctcaaaaaaaaaaaaaattgtccttgtagagaaagggccctcttgattgcaaattgttcttgttgagagagggccctcttgattgtcattttcattggaatttgcagagaaaaactcagcctaagaagctaatattttctaaataaagtttggagaaaaatcacctgtcaagtgctggttaactaaatttcttaggaccctgtaatccttaagcatgcagtttattcagatgtcaaatatttatgttgaatttgaatcaatgtggtgtgtgtgtgtgtacatatgtatgtgtatatgtttttatttatatgtatataagaataatttttttcctttagttggtggtatggattggataatgtacatatatataatttgtgaagtgataaaagtgtatttaaataatttatttctgctgacttactcttggtttttaaaatatcacattatttaattggcctcctgttacattattggctatattgaaatctaaggactaattgtagattagtggtctttatcatatcttggtataaaatttggctgatggttgttttgctatgttttaattactgatttctagggccccgtggttatgaatgtgcttcagtttcataatgcctcctgctatggcagacatccttgacatctgggcggtggattcacagatagcatctgatggctccatacctgtggatttccttttgcccactgggatttatatccagttggaggtacctcgggaagctaccatttcttatattaagcaggtatgttaataggtataatttctatgtgtttagcatactgtgtcacttgggggaaatatcattctataatagaaaatgttaatgtaaattatatgtgataatgatagtaatgaagtgatggtaattaagaaaaattagtttatgttagctagtttgaaatgtttgtttgggttattaaaactgtcaagaagattttcttttgagttcgtctttgaaattactgtctttgagcttgtcacacttttaaaaatgtgatttttggttcattttctcttctctcctttttcttctcttccccctttccttgactcttgggggaagaaagacttttctgctgttcaccagatatttttccctcagcaggcttgccttttctattgcattagtttttaaagtaaattgatgagcaccgttgatttgtaaaaaccagcatatacatgaacttgtttttctaaactttctttctaatgtttttcaaaaagctgaacccaatgtgaatatcaggagttacggtatttttaagaaaacatatttatgatatacctctgaaaactcttggctttggacaagcctcttaactcctttccaagcttcttcattagagaaactggaattctaaaatggcatgatccttaaagtaaaaagaaaatacagaattgacttaagcatctcatacgctcatgcatggtgcacctatttttatagtcaccagtgacaagtatggattcctatggctatacttcacattagattaaccatttatctctttttaattgtgaaatatacataacctggaatttactatttagacatttttaggtgtgcattcacagtgctgtgcaaccatcaccagtatccatttccagcacttaaccatcatcccaaacagaaacttcatacccataactgcccctttccctcaattcctttttatttctattctgttttccatctctaccagtttgcctattcttggtactttatataagtggaaacatacaataaatagctatccttttgtagttggcttgcatagcatagtgtttttaaagttcatccatgttgtagcatatatcctagtttcattcccttttatggctgaataatattccattgtatgtatatgccacattttgtatatttatctgttgatgaacatttgggttgcttccagcttttggctattgtgaatattatgctactctgagcattggtgtataaatgtctgtttgaattcctgctttcagttcttttgggtatatacctagaagtgtgattgttggttcacagtggtaattccatgttttaacttttgaggaactgccacactgttttccacagtggctgcaccatataatgttgggaccagcagtgtacaagggttccagtttgtctacatactggccaacacatgctattttctcctcgctccctccctccctctcttcctccctccctctcttcctccctccctctcttcctccctccctccctcttttcctacctcccctccttcctctccctctcctcccttcccctcccctctctccctccctcccttcttccttcctttcttccttcctcttttcttacattatttctttcctttttttgtttgtctaagagatacgatcttgtttgccatattttctgtgtgtgtgtgtgtgtgtgtgtgtgtgtgtgtgtgtgtgtgtgtttaatagtatccatcctaatggatgtgaagtggtatctcattgtgggtttttttttgttttttgttttgtttttttgagacggagtctcgctctgtcgccagactggagggcagtgtcatgaccttggcttactgcaacctccgcctcctgggttcaagtgattctcctgcttcagcatcctgagtagctgggactataggtgcacaccaccatgcccagctaatttttgtatttttagtagagatgggatttcaccatgttggccaggatggtcttgatttcttgacctcatgatctgcccaccatggcctcccaaagtgctgggattacaggtgtgagccactgcacccagcctcattgtggtttttatgtgcatttccctcatgactaatgatgttgagcatctttttatgtgcttgttggtcatttgtatgttttttggagaattatcttttctagacctgtactgatttttaaactttgttttctgttattgagttgtaggagttctttatatattctggatattaatatcttgtcagatatatatgatttgcaaatattacctcccattttgttggttgccttttcattctcttgataatgtcttttgctgtacagaagttttaagttttggtgaagttcagtttaccttttttttcttaatgttactattttgagcctttagaatgaaaagtttacctatcattattattattgttgttgcttgtactttctcctgtatgttttatgtaactgaatctgagctgaggattggccacaatttttaactcaggtctgttattttcagctcttagcttttgtactggctgttaacaaaatatgcaatccctttttgtttttgtgtggtgaacttcttttatattatctcatccaggagacatggaaagagatgttgagagattgattatgatcattctgacaggacagtgttagtaaggatacccctgataaaagaaaagagtgcaggacaaaaataatttatatattgtatggaggtggtgatacatactttaaattaaaattttttttgattactcctgatcagtagtttctgcttacaggaaagtgaaatatgaaagttttcattgagtcttacttgtcatagataatttgcttttagtaatttcatttataatgcattttttgtgcactatgccattatattaccctacaaacatgggataataccatatatactctcctataatttgctgttgtaagaaaacttgaatattttgtatggttttccatttcagaatgaatttatatttttttctatttttctgaatatatttctattattttcaccgttctgtaatattctataattcatttaaccaatcttttattgctaaacatttgatttgttcatagttttttatttttacaaacagtgctgcagtgagcatccttatatctttgtacacttgagcaagtgtttccattccagaaaatgtcttattttttctcatagatgttatggaagcaagttcacaattacccaatgttcaacctccttatggatattgactcctatatgtttgcatgtgtgaatcagactgctgtatatgaggagcttgaagatgaaacacgaagactctgtgatgtcagaccttttcttccagttctcaaattagtgacaagaagttgtgacccaggggaaaaattagactcaaaaattggagtccttataggaaaaggtaaattaaaaaataaaaaaaaagtacaaattcttaatatatctatttatataaacatatagtaagtatatttaagacattgcttttcaaagtgtggtccatggaccactgctgatctgtaaattgttgccagtttgagataaggtaagtatagaaattgagaataaatgtttagaaacccctttagcaatatgatattgccatagcatccaagggcttatgtttagtatatgtttttgttgtctaatgatttattatttatttaatttttaaaaatagagacagagtctcactgtgttgtccaggctggtctcaaactcctgggctcaagtgatcctcctaccttggccccccaaagtgctgggattacaggtatgagccactgtgcatggctgatttatttttattttacaaaaatattgatctttgatggattggaaagaaaaaagttgttgctttacctcagattgtttgagaagtccttgactaaaggagacattatccttagtttcttatgaactgctcctatagaatgaacatattttatacacaactctctatgccaaccaagactttaagctgtttgatgtttccataaataaattattgacaaattttacttcttaactgaaattatctttaagtagaagttattttggtagagagtcaggaaatatcatgtccacattattccttgcaaatttattccatggaaaattgtgagtaacacaatcaagaacatggaggagtctctcttgctatataacattaggagtctcaggggctaatgttttatcaatatcttgctttagagagggtactgacttgaattaggaactacaaaaggttactgaggaggggactttatagtttaaatacaaaaccagaaaatccactggagatttatgtatctagattgggtacctgtattgatcagattcactgttataggataatatagtaccatcccctttaaaatactattctttgatcttaaccctataataattcaggctagctaatgattatactgtgggactactccctctgagtgcatagaaccttgttagtttactaagggatactctgatatttaaaaatatttgattacttcagagggtggctagtttatttttggcaataaatttttcccagttaacaagtttatagttatgtatcatacaaaattttttcttatatattagtgacatataattagaaagcattattagaaagcatgcctttctattttctagagctacaaaggtgtgttttgttttgtttgttttttgtttctgtttttgtttttttggtgacaaggtctcgccctgttgcccaggctagagtgcagtggtacgatcatggctcacttcagccttgactcgaccctcagtcgcaagcgatcctcccacctcagcgtcctaagtagctgggactacatacagatgcaagccaccatgcctggctaatttttgtatttttattatagtagagacagggtgttgcccaggctggtctcaaactcctggactcgagctgtctgcctgcctcagccttcagagccacagcacctggcccaaatatttttatagaatttaaatttttgtccattataaaaggttttccctttgttctcatgcccataaaaagttgaagaatatatggaagaggagaaagaaagaaaaaaaatcacttatagtcttatcatccaggacaagtatattgaccgacaatattttgatatatttcttttgaataaattatatgtatttcaaggcttttatttgtttttgtttctgtggattaaaatacttagcttttctgtattctcatgctaatgtaagaaagcaaaccagaaccacattgatttttagggaagctaaaatatttgttaatccaaaaagtaccacagagtaggccatgttctttttgatttttttttcttttttaaatagggtctctctctgtcacctaggctggagtgcagtggtgcgatctcagctcactgcagcctcaacctcccaggcttaagtgatc

SEQ ID NO: 5 - DNA, 22 bp, artificial sequence
  primer sequence p110-359L
cctgtcaagtgctggttaacta

SEQ ID NO: 6 - DNA, 22 bp, artificial sequence
  primer sequence p110-359U
caatccataccaccaactaaag

SEQ ID NO: 7 - DNA, 42 bp, Artificial Sequence
  sense primer sequence
cggggtaccaatcacctgtcaagtgctggttaactaaatttc

SEQ ID NO: 8 - DNA, 40 bp, artificial sequence
  antisense primer sequence
gaagatctggcattatgaaactgaagcacttacataacca

(19) United States
(12) Patent Application Publication
(10) Pub. No.: US 2005/0118622 A1
(43) Pub. Date: Jun. 2, 2005

(54) Methods for predicting development of insulin resistance

(76) Inventor: Pierre Bougneres, (US)

Correspondence Address: PFIZER INC., PATENT DEPARTMENT, MS8260-1611, EASTERN POINT ROAD, GROTON, CT, 06340, US

(21) Appl. No.: 10/951,579

(22) Filed: Sep. 28, 2004

Related U.S. Application Data
(60) Provisional application No. 60/507,226, filed on Sep. 29, 2003.
(60) Provisional application No. 60/587,333, filed on Jul. 13, 2004.

(51) Int. Cl. C12Q 1/68; A61K 31/4439; A61K 31/426

(52) U.S. Cl. 435/6; 514/369; 514/341

(57) ABSTRACT

Methods of predicting a subject's likelihood of developing insulin resistance comprise determining in a subject the identity of the nucleotide present at position 100 of SEQ ID NO 1, wherein the allele comprising said nucleotide is correlated with an increased or decreased likelihood of developing insulin resistance, thereby identifying the subject as having an increased or decreased likelihood of developing insulin resistance. Methods for treating or preventing insulin resistance in a subject are also disclosed.

This invention relates to compositions and methods for predicting insulin resistance, particularly in obese subjects. Also provided are methods for treating individuals and for the clinical testing of medicaments involving carrying out a diagnostic assay capable of predicting insulin resistance, particularly obesity-linked insulin resistance, and methods for identifying agents capable of treating or preventing insulin resistance.

Insulin resistance is defined as a smaller than expected biological response to a given dose of insulin. Insulin resistance usually refers to resistance to the effects of insulin on glucose uptake, metabolism, or storage, and is a ubiquitous correlate of obesity. Indeed, many of the pathological consequences of obesity are thought to involve-insulin resistance. These include hypertension, hyperlipidemia and, most notably, non-insulin dependent diabetes mellitus (NIDDM). Type II diabetes is a heterogenous group of disorders in which hyperglycemia results from both impaired insulin secretory response to glucose and decreased insulin effectiveness or sensitivity (i.e., insulin resistance). Most NIDDM patients are obese, and a very central and early component in the development of NIDDM is insulin resistance (reviewed in Moller et al., New. Eng. J. Med. 325:938, 1991).

Epidemiological studies in different populations have shown that peripheral insulin resistance begins a progression of type 2 diabetes over a continuum of worsening insulin action that ends with the loss of insulin secretion. The ensuing dysregulation of carbohydrate and lipid metabolism that occurs as a consequence of insulin resisitance further exacerbates its progression. Beta cells of the pancreas normally compensate for the insulin resistance state by increasing basal and postprandial insulin secretion. At some point, the beta cells can no longer compensate, failing to respond appropriately to glucose. Insulin resistance in obesity and type 2 diabetes is manifested by decreased insulin-stimulated glucose transport and metabolism in adipocytes and skeletal muscle and by impaired suppression of hepatic glucose output. These defects can result from impaired insulin signaling in all three target tissues, as well as from downregulation of the major insulin-responsive glucose transporter GLUT4 in adipocytes.

Obesity results from an imbalance between caloric intake and energy expenditure. As adipose tissue plays a crucial role in the regulation of energy storage and mobilization, it has been the focus for studies directed to identifying candidate genes and finding abnormalities in adipocyte physiology or metabolism (Plata-Salaman, Brain Behav. Immun. 3:193, 1989; Lardy et al., Annu. Rev. Biochem. 59:689, 1990). Potential candidate genes for obesity have included those that influence energy expenditure such as the human uncoupling protein, a molecular marker widely regarded as being specific for brown adipose tissue, suggesting that modulation of thermogenesis in adipose tissue may also be important in humans (Cassard, et al., J. Cell Biochem., 43:255-264, 1990). Studies on twins, adopted children and on animal models of obesity have shown that genetic factors are implicated in the dynamics of gaining weight (Bouchard, C., Perusse, L., Ann. Rev. Nutr., 8:259-277, 1988).

There is a need to identify genes and defects in those genes that are related to insulin resistance. Such genes are useful as molecular markers for the early detection of susceptible individuals so that intervention regimes may be instituted for delay or prevention of insulin resistance and type 2 diabetes.

Phosphoinositide 3-kinases (PI3Ks)

Phosphoinositide 3-kinases (PI3Ks) are enzymes that phosphorylate the D-3 position of phospholipids containing an inositol headgroup (phosphoinositides). PI3Ks are involved in many cellular responses triggered by external stimuli. For example, insulin-dependent glucose uptake is thought to require PI3K activation. Several classes of PI3Ks exist in mammalian cells. Class IA PI3Ks are heterodimers of a catalytic subunit of about 110 kDa (p110) and a regulatory subunit, usually of about 85 kDa (p85). The p110 proteins contain a kinase domain at the C-terminus, and a p85 and ras binding domains at the N-terminus.

Three genes encoding regulatory subunits have been identified in mammals. All contain two SH2 domains and an inter-SH2 domain containing sequences allowing the interaction of the regulatory and catalytic subunits. Two isoforms, p85α and p85β, generate products of 85 kDa. Both of these isoforms contain an SH3 domain, a Bcr/Rac GTPase-activating protein (GAP) homology (BH) domain, and two proline-rich regions (P1 and P2) located on either side of the BH domain, in addition to the SH2 domains. Three splicing variants are known, including a form arising from an insertion of 8 amino acids in the inter-SH2 domain adding two potential serine phosphorylation sites. The other two are truncated versions of p85α with apparent molecular masses of 55 kDa, termed AS53 or p55α and 46 kDa termed p50α. These lack the SH3, P1 and BH domains, and are thought to arise from alternative transciption start sites with generate short novel sequence at the N-terminus. P55α is highly expressed in brain and muscle and p50α is highly expressed in brain, liver, muscle and kidney. A third PI3k adapter subunit gene has been characterized, known as p55PIK or p55γ, producing a protein of 55 kDa which is highly homologous in sequence and has an identical domain structure to the p55α splice variant of p85α. The 32-amino acid N-terminal regions of p55α and p55γ are closely related, but their function is currently unknown. The 32-amino acid domain contains a YXXM motif which, if phosphorylated, could potentially form intramolecular interactions of links to other SH2 domain-containing proteins.

The insulin receptor tyrosine kinase is activated by binding of insulin to the extracellular region of its receptor. The activated tyrosine kinase phosphorylates IRS proteins on numerous phosphotyrosine (pTyr) residues. Some of these are specific binding sites for the SH2 domains of class 1A regulatory subunits. Association of PI3K with IRS proteins increases the lipid kinase activity of the p110 subunit and brings it into proximity with substrates at the membrane. The lipid products act as second messengers to recruit other signaling proteins to the membrane. This signaling eventually leads to glucose uptake by the cell. The importance of PI3K in this signaling process is supported by two general types of experiments. First, compounds that inhibit p110 kinase activity (e.g., wortmannin, Ly294002) block insulin-mediated glucose transport in cultured cells. Second, expression of constitutively active forms of PI3K can stimulate glucose transport and dominant negative forms can inhibit glucose transport.

The p110kDa catalytic subunit exists in four homologous forms, referred to as p100α, p110β, p110δ, and p110γ (Hiles et al, (1992) Cell 70:419-429). The α and β forms are widely expressed, while the δ form is expressed only in haematopoietic cells. Activation of PI 3k in response to insulin increases the translocation of GLUT4 to the plasma membrane and causes a subsequent stimulation of glucose transport. The P110β form is described in Hu et al, (1993) (Mol. Cell. Biol. 13: 7677-7688). More recently, Kossila et al, (2000) (Diabetes 49: 1740-1743) cloned the p110β gene and screened the 22 exons, the introns and 1.5 kb of the proximal/5′ region for polymorphisms.

The present invention is based on the discovery that human subjects carrying an allele at a polymorphism in the 5′ region of the p110β catalytic subunit of the phospatidylinositol (PI) 3-kinase gene are less likely to suffer from or develop insulin resistance than subjects not carrying said allele. It was shown in particular that obese subjects carrying a p110β allele are less likely to develop, or protected from developing, insulin resistance. The inventors further demonstrated that a p110β allele associated with protection from developing insulin resistance results in increased expression of the p110β. Modulating p110β levels or activity may therefore be useful for conferring protection or preventative treatment for insulin resistance in subjects in need thereof.

The present invention relates to the identification of a p110β subunit as an important factor contributing to differences in insulin resistance, particularly in obese individuals. The invention provides a method to predict whether a subject is more or less susceptible to developing insulin resistance.

Information regarding the likelihood of a subject to have or develop insulin resistance allows a treatment to be adapted for a particular subject. The invention provides economic benefits and/or improved therapeutic outcome since a subject can be treated earlier and/or more aggressively for insulin resistance and/or obesity, thereby preventing or delaying a subject's development of insulin resistance or progress to type 2 diabetes.

In another aspect, the invention discloses a method of predicting whether a subject, preferably an obese subject, is suffering from or susceptible to developing insulin resistance, the method comprising determining in the subject the presence or absence of an allele of a gene encoding a subunit of the PI 3k protein, or an allele in linkage disequilibrium therewith, wherein the allele is correlated with a likelihood of having an increased or decreased susceptibility to developing insulin resistance, thereby identifying the subject as having an increased or decreased likelihood of suffering from or developing insulin resistance. In one aspect, said gene encoding a subunit of the PI 3k protein is a gene encoding a p110β subunit.

In a preferred aspect, the invention discloses a method of predicting whether a subject, preferably an obese subject, is suffering from or susceptible to developing insulin resistance, comprising determining in the subject the presence or absence of an allele of the p110β gene, or an allele in linkage disequilibrium therewith, correlated with a likelihood of having an increased or decreased susceptibility to developing insulin resistance, thereby identifying the subject as having an increased or decreased likelihood of suffering from or developing insulin resistance.

Linkage disequilibrium may be assessed using any suitable method, for example using the methods described in U.S. Pat. No. 6,476,208, the disclosure of which is incorporated herein by reference.

The methods may further comprise administering an effective amount of an agent for the treatment or prevention of insulin resistance to said subject if the subject is identified as having an increased likelihood of developing insulin resistance.

The invention also encompasses a method of predicting a subject's likelihood of developing insulin resistance, comprising determining whether the subject has an increased or decreased level of p110β expression, wherein increased or decreased level of p110β expression is correlated with an increased or decreased likelihood of developing insulin resistance, thereby identifying the subject as having an increased or decreased likelihood of developing insulin resistance. The method may further comprise administering an effective amount of an agent for the treatment or prevention of insulin resistance to said subject if the subject is identified as having an increased likelihood of developing insulin resistance.

Another embodiment encompasses a method for treating or preventing insulin resistance in a subject, the method comprising:
(a) determining in the subject the presence or absence of an allele of the p110β gene, wherein the allele is correlated with an increased or decreased likelihood of developing insulin resistance; and
(b) selecting or determining an effective amount of an agent for the treatment or prevention of insulin resistance to administer to said subject. The method may further comprise (c) administering said effective amount of said agent to said subject.

Also disclosed is a method for treating or preventing insulin resistance in a subject, the method comprising:
(a) determining whether the subject has an increased or decreased level of p110β expression, wherein an increased or decreased level of p110β expression is correlated with an increased or decreased likelihood of developing insulin resistance; and
(b) determining an effective amount of an agent for the treatment or prevention of insulin resistance to administer to said subject. Preferably, the method further comprises
(c) administering said effective amount of said agent to said subject.

Also provided is a method of identifying a test compound that modulates p110β transcription, said method comprising: comparing the level of transcription from a p110β regulatory region in the presence and absence of a test compound wherein a determination that the level of transcription is increased or decreased in the presence of said test compound relative to the level of transcription in the absence of said test compound indicates that said compound is a candidate modulator of transcription. Preferably, a determination that the level of transcription is increased or decreased in the presence of said test compound relative to the level of transcription in the absence of said test compound indicates that said compound is a candidate compound for the treatment or prevention of insulin resistance. In one example, the level of transcription from said p110β regulatory region in the presence and absence of the test compound is determined by performing an in vitro transcription reaction using a construct comprising said p110β regulatory region. In another example, the level of transcription from said p110β responsive regulatory region in the presence and the absence of the test compound is determined by measuring the level of transcription from a p110β regulatory region in a cell. Said p110β regulatory region may comprise a contiguous span of at least 30 nucleotides of SEQ ID No 1 or the complements thereof, or homologs thereof having at least 60%, 70%, 80%, 90%, 95 or preferably 98% or 99% nucleotide identity thereto.

In preferred aspects of the methods of the invention, the subject is an obese human subject, the subject preferably having a BMI of between about 25 and 50. Preferably, the subject has a BMI of at least 25, 30, 35, 40 or 45.

The invention also provides a method of predicting a subject's response to a therapeutic agent, said method comprising: determining in the subject the presence or absence of an allele of the p110β gene, wherein the allele is correlated with an increased or decreased susceptibility to developing insulin resistance, thereby identifying the subject as having an increased or decreased likelihood of having a positive response to treatment with said agent, or more preferably increased or decreased susceptibility to developing insulin resistance. In one embodiment, said therapeutic agent is an agent for the treatment or prevention of obesity. In another embodiment, said therapeutic agent is an agent for the treatment or prevention of insulin resistance.

The methods of the invention can also be used particularly advantageously in methods of treatment. In one example, the invention provides methods to determine the course of treatment for an individual. A determination that an individual has an increased susceptibility to insulin resistance indicates that preventative treatment for obesity or insulin-resistance may be beneficial. In another aspect, the methods of the invention are used to determine the amount of a medicament to be administered to a subject. In yet another example, methods of the invention are used to determine a suitable medicament to be administered to a subject, preferably an obese subject. In another example, the methods are used to assess the therapeutic response of subjects in a clinical trial or to select subjects for inclusion in a clinical trial. For instance, the methods of the invention may comprise determining the genotype of a subject at the p110β gene, wherein said genotype places said subject into a subgroup in a clinical trial or in a subgroup for inclusion in a clinical trial.

In one aspect of any of the methods of the invention, the step of determining whether the DNA of subject comprises a particular p110β allele can be performed using a nucleic acid molecule that specifically binds a p110β nucleic acid molecule. Preferably, the p110β allele comprises detecting a polymorphism in a transcriptional regulatory region. In further preferred aspects, the methods of the invention comprise determining whether the DNA of an individual comprises a T or a C at position 100 of SEQ ID NO 1, or at position −359 (position 359 upstream from the start codon) of the p110β gene. This may thus comprise determining whether the genomic DNA of an individual comprises a p110β allele, whether mRNA obtained from an individual comprises a p110β allele.

Preferably, in any of the above embodiments, determining whether the DNA of an individual comprises a p110β allele may comprise the steps of:
a) providing a biological sample;
b) contacting said biological sample with: a polynucleotide that hybridizes under stringent conditions to a p110β nucleic acid, preferably to a p110β nucleic acid comprising position 100 of SEQ ID NO 1, or position −359 (position 359 upstream from the start codon of the p110β gene); and
c) detecting the presence or absence of hybridization between said polynucleotide and an RNA species within said sample.

Preferably the biological sample is contacted with a polynucleotide that hybridizes under stringent conditions to a p110β nucleic acid, wherein detection of said hybridization or of said binding indicates that said p110β is expressed within said sample.

Preferably, said polynucleotide is a primer, and wherein said hybridization is detected by detecting the presence of an amplification product comprising said primer sequence. Detecting the p110β nucleic acids can be carried out by any suitable method. Oligonucleotide probes or primers hybridizing specifically with a p110β genomic or cDNA sequence are also part of the present invention, as well as DNA amplification and detection methods using said primers and probes.

In yet further aspects, the invention encompasses methods for identifying ligands that modulate the expression of the p110β gene.

In the methods of the present invention, said allele is comprises a sequence of SEQ ID NO 1, or a variant (e.g. deletion, insertion, substitution) thereof, and complements thereof. In preferred aspects, the allele of the p110β gene is located in the 5′ regulatory region of the p110β gene. More preferably said allele is a a polymorphism of the p110β gene described in Kossila et al, (2000) (Diabetes 49: 1740-1743), the disclosure of which is incorporated herein by reference. In a most preferred aspect, said method comprises determining the nucleotide present at position 100 of SEQ ID NO 1, or at position −359 (position 359 upstream from the start codon) of the p110β gene.

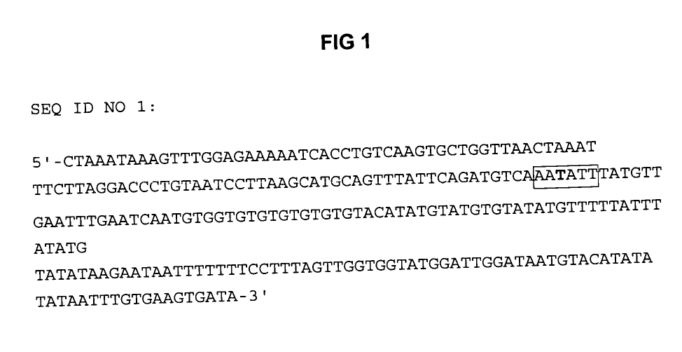

FIG. 1 shows a genomic DNA sequence comprising a portion of the proximal 5′ region of the p110β gene (SEQ ID NO: 1). The polymorphic base at position 100 of SEQ ID NO 1, which corresponds to position −359 (359 upstream of the start codon) of the p110β gene, is shown in bold. The polymorphic base was either a T or a C.

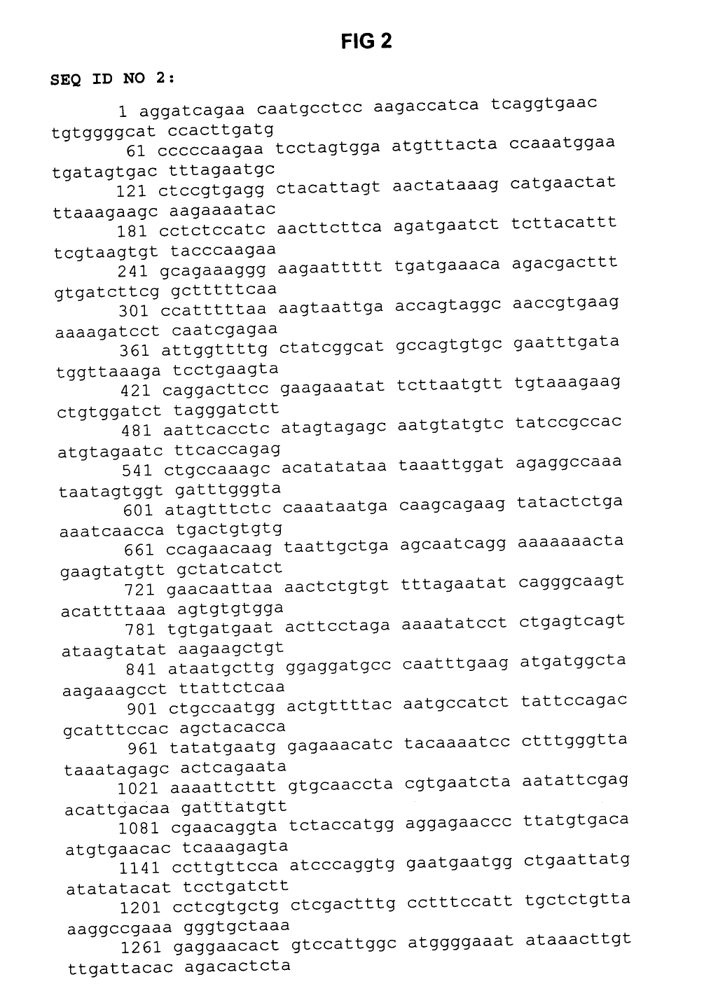

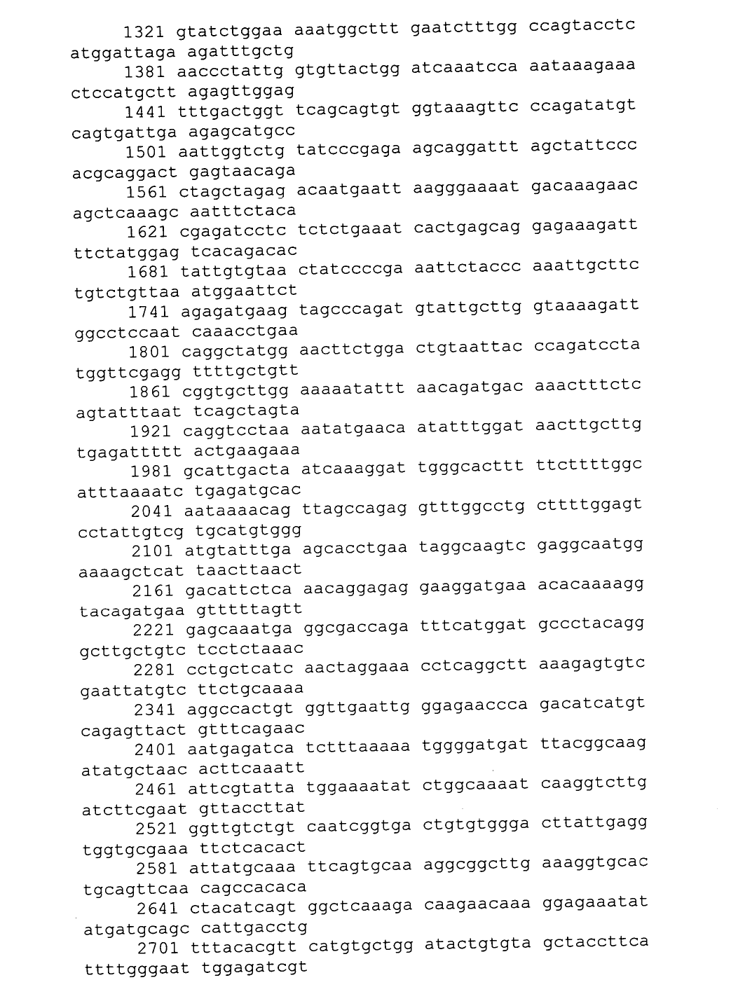

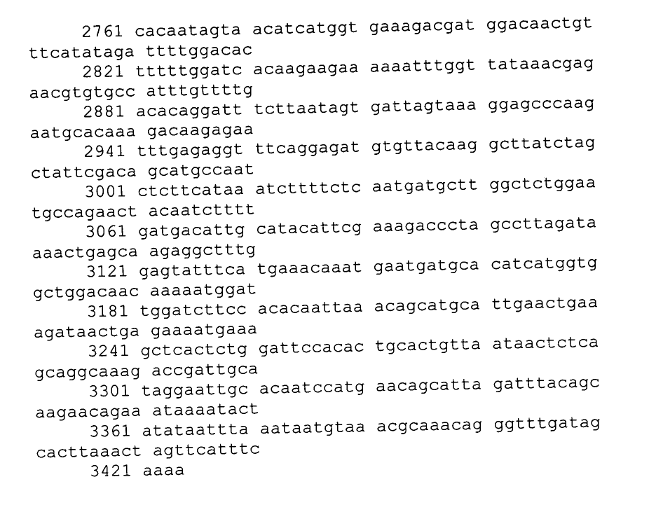

FIG. 2 shows the cDNA sequence (SEQ ID NO 2) encoding the p110β polypeptide as shown in Genbank accession number Z29090.

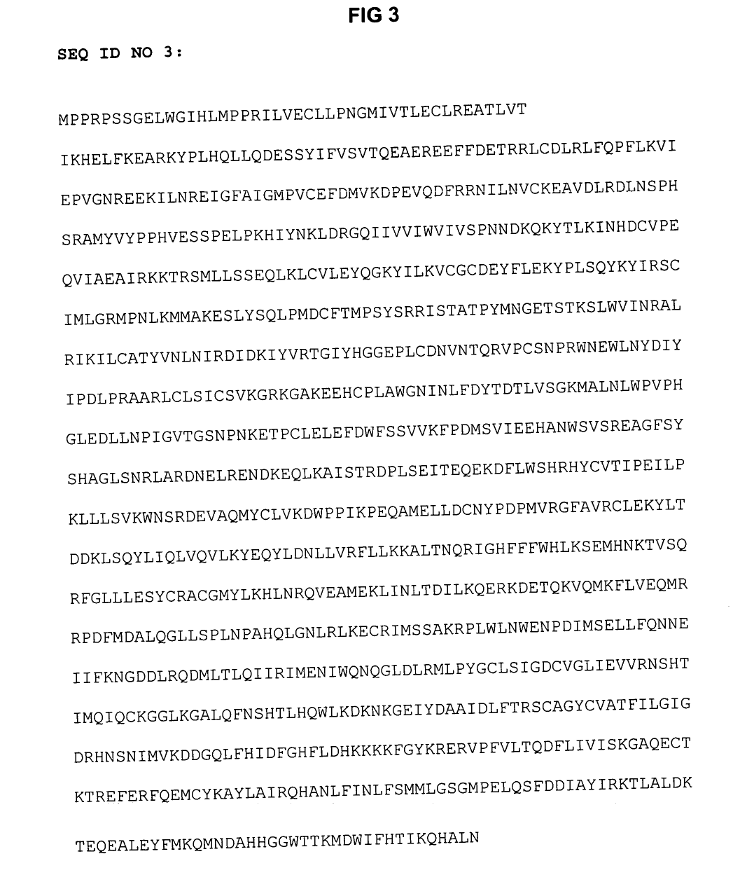

FIG. 3 shows the amino acid sequence (SEQ ID NO 3) of the p110β polypeptide as shown in Genbank accession number Z29090.

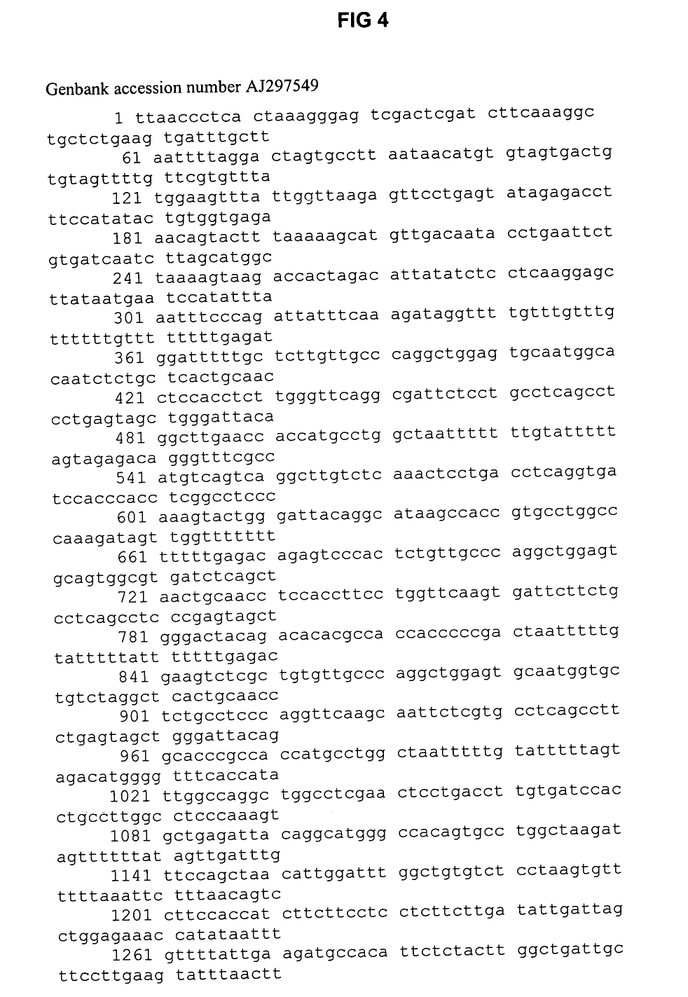

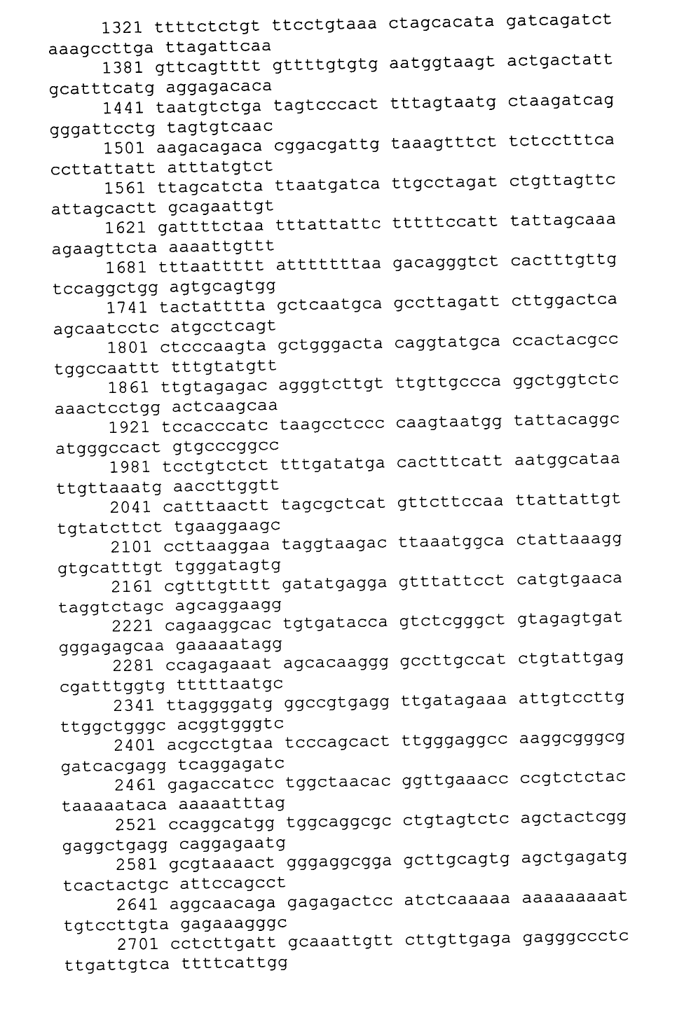

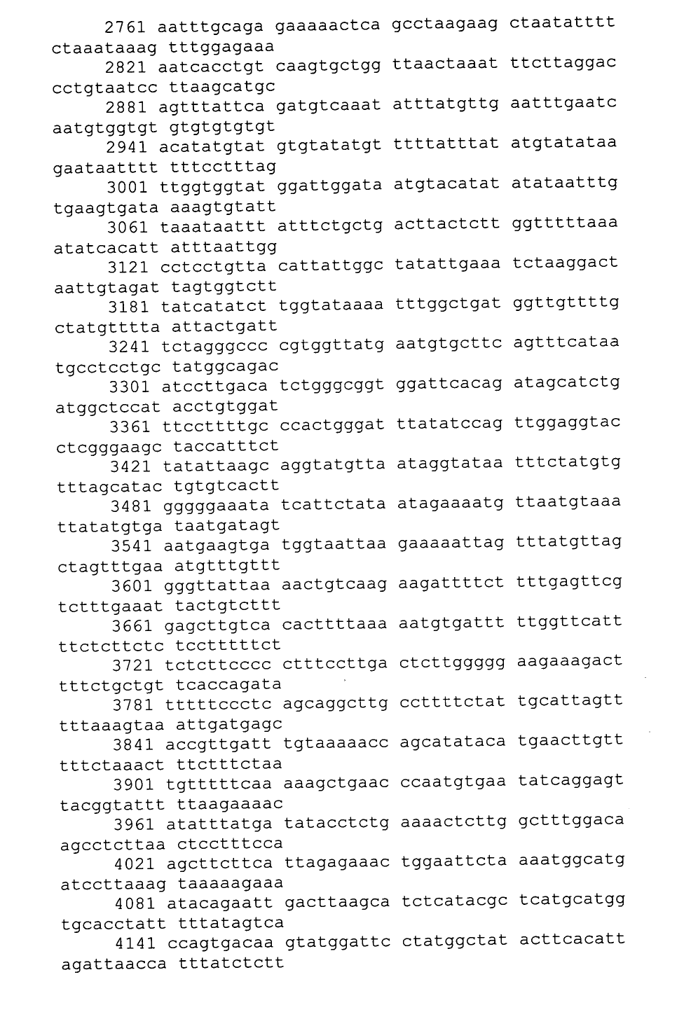

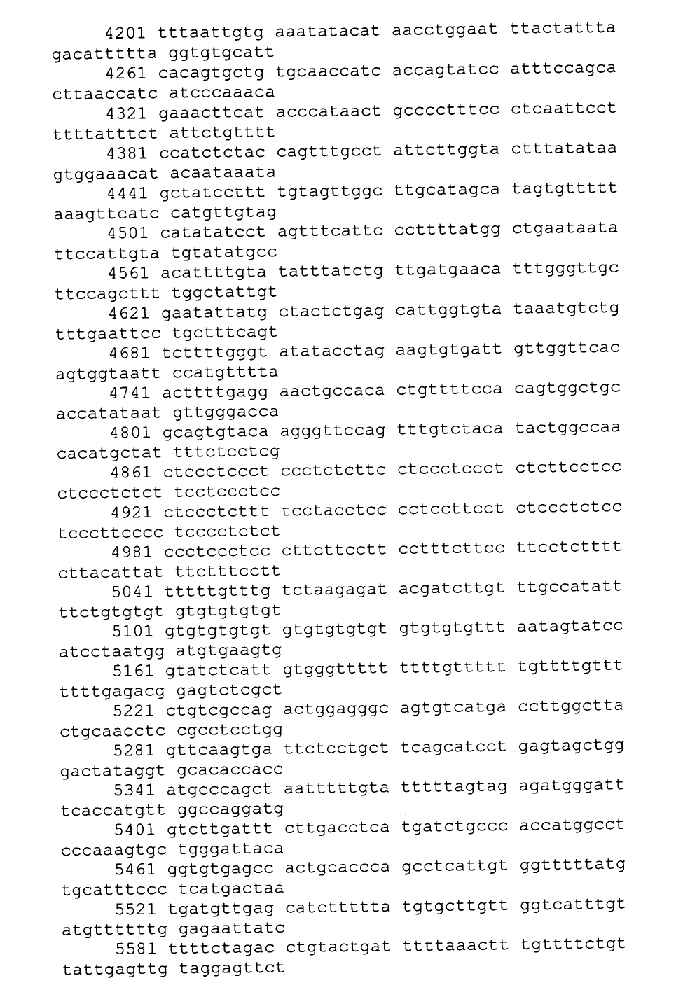

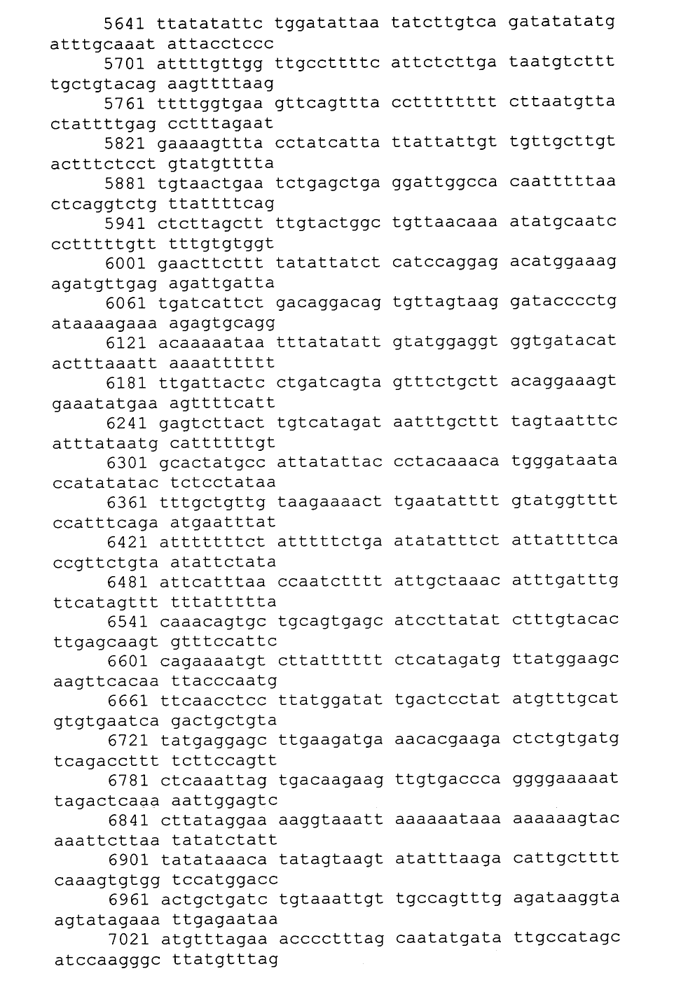

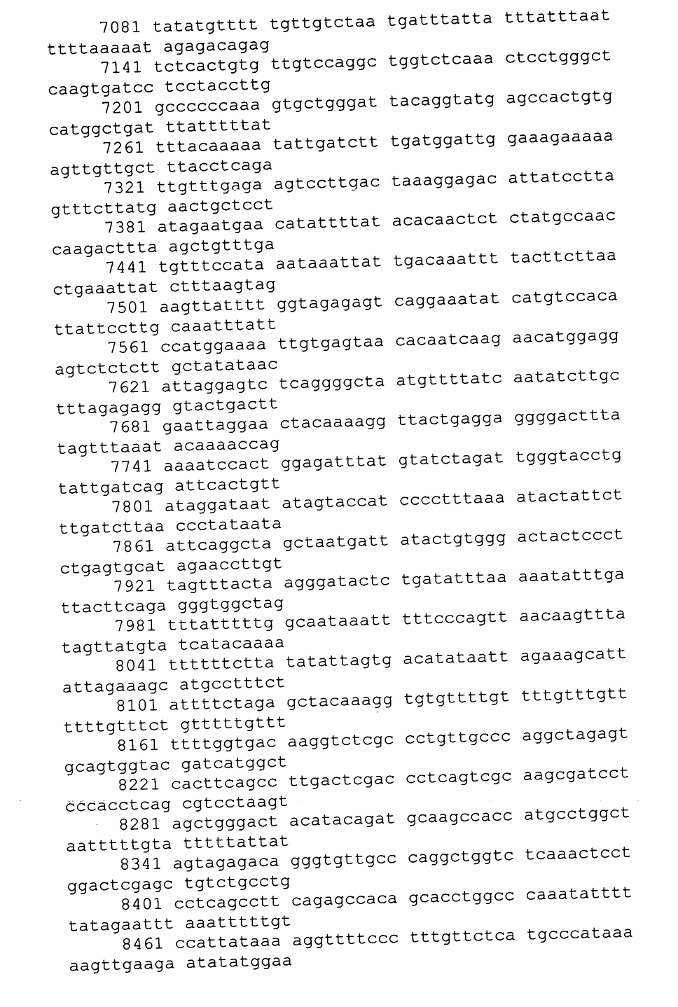

FIG. 4 shows a portion of the genomic DNA sequence (SEQ ID NO 4) encoding the p110β polypeptide from Genbank accession number AJ297549. The sequence shown in FIG. 4 includes a portion of the proximal 5′ regulatory region of the gene as well as exons 1 and 2 and introns 1 and 2.

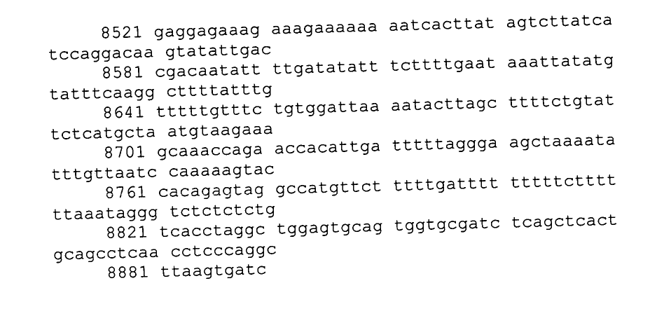

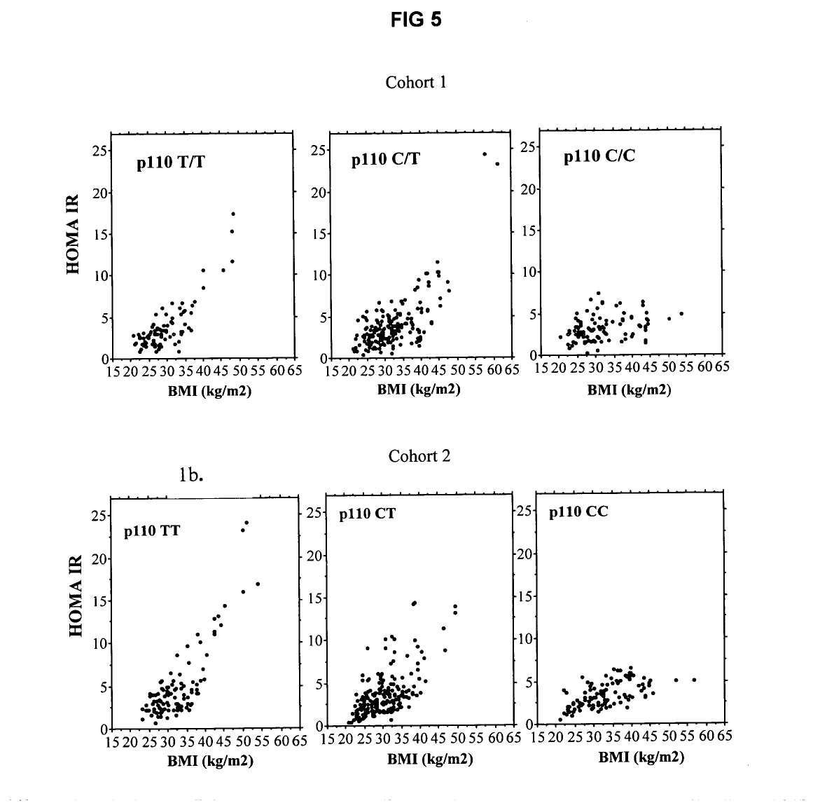

FIG. 5 shows insulin resistance in obese subjects as a function of BMI in each of (a) TT or TC and (b) CC genotype groups for the two cohorts of patients tested. The upper row of graphs represents the first cohort, the second row of graphs the second cohort. Plotted on the x-axis is BMI (kg/m2) and insulin resistance index (HOMA-IR) on the y-axis. The figure shows that obese subjects carrying the CC genotype had lower insulin resistance than TT or TC genotypes when comparing individuals with a similar BMI.

Three hundred thirty eight children suffering from obesity were examined for association of the polymorphism at position −359 (359 nucleotides upstream of the start codon) of the p110β gene with insulin response. Either a T or a C is commonly present at position −359 of the p110β gene. Genotyping was carried out using PCR-based methods; nucleotide positions 2826 to 3116 bp of the p110β nucleotide sequence shown in Genbank accession number AJ297549 were amplified, said nucleotide positions corresponding to nucleotide positions −436 to −246 bp with respect to the ATG of the p110β gene. Patients having a higher BMI generally have higher insulin resistance. However, when adjusted for BMI, patients in the CC group had lower insulin resistance values than patients in the TT and TC groups, particularly at higher BMIs, showing that patients homozygous for the C allele are less likely to develop insulin resistance compared to TT and TC patients. Results are shown in FIG. 5.

The T→C change at position −359 of the p110β gene creates a binding site for the GATA2 and GATA3 transcription factors (a TAGATAT sequence is present on the complementary DNA strand). GATA2 and 3 are expressed highly in pre-adipocytes (Tong et al, Science 290:134-138 (2000) and are known to control the transition from pre-adipocyte to adipocyte. In order to investigate whether the polymorphism at position −359 of the p110β gene affects the level of p110β expression, transient transfection assays were carried out in which fragments of human p110β promoter carrying either the −359T or −359C allele were linked to a luciferase coding sequence and transfected in NIH-3T3 cells. The assay showed that the −359C allele resulted in significantly increased expression from the p110β promoter fragment. When combined with the association of the −359C allele with decreased likelihood of developing or suffering from insulin resistance, the results suggest that increasing p110β expression may confer protection from developing insulin resistance or may be useful in treating an individual suffering from insulin resistance.

As discussed above, the present invention pertains to the field of pharmacogenomics and predictive medicine in which diagnostic assays, prognostic assays, and monitoring clinical trials are used for prognostic (predictive) purposes to thereby treat an individual. Accordingly, one aspect of the present invention relates to diagnostic assays for determining p110β protein and/or nucleic acid expression, in the context of a biological sample (e.g., blood, serum, cells, tissue) to thereby determine whether an individual is susceptible to or suffering from insulin resistance. Such assays can be used for prognostic or predictive purpose to thereby phophylactically treat an individual prior to the onset of a disorder characterized by or associated with insulin resistance, for example by administration of an effective amount of a insulin sensitizing drug such as a thiazolidinedione (TZD). The invention also pertains to methods of treating an individual by increasing p110β expression as well as to method for identifying candidate therapeutic molecules capable of increasing p110β expression.

Because the present inventors have discovered alleles of the p110β are associated with increased or decreased expression of p110β gene, the methods of the invention may be useful also to detect whether an individual is afflicted with a disease or disorder, or is at risk of developing a disorder, associated with increased or decreased p110β activity.

Furthermore, based on the discovery that alleles of the p110β is associated with increased or decreased expression of p110β gene are also associated with protection from or susceptibility to insulin resistance, the inventors provide that p110β expression levels may be used to assess susceptibility to insulin resistance. In further aspects, the invention provides methods for screening compounds for the identification of agents capable of modulating, preferably increasing, the level of p110β expression. Such agents may be useful in the treatment or prevention of insulin resistance.

Definitions

The term “agent” is used herein to denote a chemical compound, a mixture of chemical compounds, a biological macromolecule, preferably a peptide or protein, or an extract made from biological materials such as bacteria, plants, fungi, or animal (particularly mammalian) cells or tissues.

In the context of the present invention, a “positive response” or “positive therapeutic response” to a medicament or agent can be defined as comprising a reduction of the symptoms related to a disease or condition. In the context of the present invention, a “negative response” to a medicament can be defined as comprising either a lack of positive response to the medicament, or which leads to a side-effect observed following administration of a medicament.

The term “polypeptide” refers to a polymer of amino acids without regard to the length of the polymer; thus, peptides, oligopeptides, and proteins are included within the definition of polypeptide. This term also does not specify or exclude post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring. The term “recombinant polypeptide” is used herein to refer to polypeptides that have been artificially designed and which comprise at least two polypeptide sequences that are not found as contiguous polypeptide sequences in their initial natural environment, or to refer to polypeptides which have been expressed from a recombinant polynucleotide.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame.

The term “primer” denotes a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified.

As used herein, a “test sample” refers to a biological sample obtained from a subject of interest. For example, a test sample can be a biological fluid (e.g., serum), cell sample, or tissue.

The term “genotype” as used herein refers the identity of the alleles present in an individual or a sample. In the context of the present invention a genotype preferably refers to the description of the alleles present in an individual or a sample. The term “genotyping” a sample or an individual for an allele involves determining the specific allele carried by an individual.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A polymorphism may comprise a substitution, deletion or insertion of one or more nucleotides. A single nucleotide polymorphism is a single base pair change.

The term “allele” is used herein to refer to a variant of a nucleotide sequence. For example, alleles of the p110β nucleotide sequence include C and T alleles at nucleotide position 100 of SEQ ID NO 1, or the p110β −359T and −359C alleles.

As used herein, “exon” refers to any segment of an interrupted gene that is represented in the mature RNA product.

As used herein, “intron” refers to a segment of an interrupted gene that is not represented in the mature RNA product. Introns are part of the primary nuclear transcript but are spliced out to produce mRNA, which is then transported to the cytoplasm.

The term “p110β gene”, when used herein, encompasses genomic, mRNA and cDNA sequences encoding any p110β protein, including the untranslated regulatory regions of the genomic DNA. The p110β gene encompasses the nucleotide sequence of SEQ ID NO 1 as well as the nucleotide sequence shown in Genbank accession number AJ297549, the disclosure of which is incorporated herein by reference, both sequences describing a portion of the proximal 5′ region of the p110β gene. The term “p110β gene” also encompasses alleles of the p110β gene, such as the p110β −359T and −359C alleles.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90% or 95% of the length of the reference sequence (e.g., when aligning a second sequence to e.g. a P110β amino acid sequence of SEQ ID NO: 3, at least 50, preferably at least 100, more preferably at least 200, amino acid residues are aligned or when aligning a second sequence to the P110β DNA sequence of SEQ ID NO: 1, preferably at least 100, preferably at least 200, more preferably at least 300 nucleotides are aligned. The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “identity” is equivalent to amino acid or nucleic acid “homology”). The percent homology between the two sequences is a function of the number of identical positions shared by the sequences (i.e., % homology=number (#) of identical positions/total number (#) of positions 100).

The comparison of sequences and determination of percent homology between two sequences can be accomplished using a mathematical algorithm. A preferred, non-limiting example of a mathematical algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77, the disclosures of which are incorporated herein by reference in their entireties. Such an algorithm is incorporated into the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the P110β sequence. BLAST protein searches can be performed with the XBLAST program, score=50, wordlength=3 to obtain amino acid sequences homologous to the P110β sequence. To obtain gapped alignments for comparison purposes, Gapped BLAST can be utilized as described in Altschul et al., (1997) Nucleic Acids Research 25(17):3389-3402. When utilizing BLAST and Gapped BLAST programs, the default parameters of the respective programs (e.g., XBLAST and NBLAST) can be used (see, www.ncbi.nlm.nih.gov, the disclosures of which are incorporated herein by reference in their entireties). Another preferred, non-limiting example of a mathematical algorithim utilized for the comparison of sequences is the algorithm of Myers and Miller, CABIOS (1989), the disclosures of which are incorporated herein by reference in their entireties. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

As used herein, the term “hybridizes to” is intended to describe conditions for moderate stringency or high stringency hybridization, preferably where the hybridization and washing conditions permit nucleotide sequences at least 60% homologous to each other to remain hybridized to each other. Preferably, the conditions are such that sequences at least about 70%, more preferably at least about 80%, even more preferably at least about 85%, 90%, 95% or 98% homologous to each other typically remain hybridized to each other. Stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are as follows: the hybridization step is realized at 65° C. in the presence of 6×SSC buffer, 5× Denhardt's solution, 0,5% SDS and 100 μg/ml of salmon sperm DNA. The hybridization step is followed by four washing steps:
two washings during 5 min, preferably at 65° C. in a 2×SSC and 0.1% SDS buffer;
one washing during 30 min, preferably at 65° C. in a 2×SSC and 0.1% SDS buffer,
one washing during 10 min, preferably at 65° C. in a 0.1×SSC and 0.1% SDS buffer,
these hybridization conditions being suitable for a nucleic acid molecule of about 20 nucleotides in length. It will be appreciated that the hybridization conditions described above are to be adapted according to the length of the desired nucleic acid, following techniques well known to the one skilled in the art, for example be adapted according to the teachings disclosed in Hames B. D. and Higgins S. J. (1985) Nucleic Acid Hybridization: A Practical Approach. Hames and Higgins Ed., IRL Press, Oxford; and Current Protocols in Molecular Biolog (supra). Preferably, an isolated nucleic acid molecule of the invention that hybridizes under stringent conditions to a sequence of SEQ ID NO 1 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

p110β in Diagnostics, Therapy and Pharmacogenetics

In preferred embodiments, the invention involves determining whether a subject expresses a p110β allele associated with an increased or decreased likelihood of developing insulin resistance or with increased or decreased p110β activity. Determining whether a subject expresses a p110β allele or determining p110β expression level can be carried out by detecting a p110β protein or nucleic acid.

Preferably, the methods of treating, diagnosing or assessing a subject comprise assessing or determining whether a subject's DNA contains a p110β allele, for example a −359T or −359C allele. The invention thus preferably involves determining whether a p110β allele is present within a biological sample comprising: a) contacting said biological sample with a polynucleotide that hybridizes under stringent conditions to a p110β nucleic acid; and b) detecting the presence or absence of hybridization between said polynucleotide and an nucleic acid species within said sample. A detection of said hybridization or of said binding indicates that said p110β allele or isoform is present within said sample. Preferably, the polynucleotide is a primer, and wherein said hybridization is detected by detecting the presence of an amplification product comprising said primer sequence.

Also envisioned is a method of determining whether a mammal, preferably human, has an elevated or reduced level of p110β expression, comprising: a) providing a biological sample from said mammal; and b) comparing the amount of a p110β polypeptide or of a p110β RNA species encoding a p110β polypeptide within said biological sample with a level detected in or expected from a control sample. An increased amount of said p110β polypeptide or said p110β RNA species within said biological sample compared to said level detected in or expected from said control sample indicates that said mammal has an elevated level of p110β expression, and wherein a decreased amount of said p110β polypeptide or said p110β RNA species within said biological sample compared to said level detected in or expected from said control sample indicates that said mammal has a reduced level of p110β expression.

An exemplary method for detecting the presence or absence of the p110β protein or nucleic acid in a biological sample involves obtaining a biological sample from a test subject and
(a) contacting said biological sample with:
   i) a polynucleotide that hybridizes under stringent conditions to a p110β nucleic acid; or
   i) a detectable polypeptide that selectively binds to a p110β polypeptide; and
b) detecting the presence or absence of hybridization between said polynucleotide and a nucleic acid species within said sample, or the presence or absence of binding of said detectable polypeptide to a polypeptide within said sample;
wherein a detection of said hybridization or of said binding indicates that p110β is expressed within said sample.

A preferred agent for detecting p110β mRNA or genomic DNA is a labeled nucleic acid probe capable of hybridizing to p110β mRNA or genomic DNA. The nucleic acid probe can be, for example, a human nucleic acid, or a portion thereof, such as an oligonucleotide of at least 15, 30, 50, 100, 250 or 500 nucleotides in length and sufficient to specifically hybridize under stringent conditions to p110β mRNA or genomic DNA. Other suitable probes for use in the diagnostic assays of the invention are described herein.

A preferred agent for detecting the p110β protein is an antibody capable of binding to the p110β protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

The term “biological sample” is intended to include tissues, cells and biological fluids isolated from a subject, as well as tissues, cells and fluids present within a subject. That is, the detection method of the invention can be used to detect candidate mRNA, protein, or genomic DNA in a biological sample in vitro as well as in vivo. For example, in vitro techniques for detection of candidate mRNA include Northern hybridizations and in situ hybridizations. In vitro techniques for detection of the candidate protein include enzyme linked immunosorbent assays (ELISAs), Western blots, immunoprecipitations and immunofluorescence. In vitro techniques for detection of candidate genomic DNA include Southern hybridizations. Furthermore, in vivo techniques for detection of the p110β protein include introducing into a subject a labeled anti-antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a serum sample isolated by conventional means from a subject.

In another embodiment, the methods further involve obtaining a control biological sample from a control subject, contacting the control sample with a compound or agent capable of detecting the p110β protein, mRNA, or genomic DNA, such that the presence of p110β protein, mRNA or genomic DNA is detected in the biological sample, and comparing the presence of p110β protein, mRNA or genomic DNA in the control sample with the presence of p110β protein, mRNA or genomic DNA in the test sample.

The invention also encompasses kits for detecting the presence of the p110β protein, mRNA, or genomic DNA in a biological sample. For example, the kit can comprise a labeled compound or agent capable of detecting p110β protein or mRNA in a biological sample; means for determining the amount of p110β protein or mRNA in the sample; and means for comparing the amount of p110β protein, mRNA, or genomic DNA in the sample with a standard. The compound or agent can be packaged in a suitable container. The kit can further comprise instructions for using the kit to detect p110β protein or nucleic acid.

Most preferably, the assays described herein can be utilized to identify a subject having or at risk of developing insulin resistance, particularly obesity-linked insulin resistance. In particular, a subject can be identified as having or at risk of developing insulin resistance if (a) the subject's DNA comprises a p110β allele associated with increased risk of developing insulin resistance, and/or (b) the subject has abnormal, particularly decreased, levels of p110β expression.

The prognostic assays described herein can be used to determine whether and/or according how a subject is to be administered a treatment for the treatment of obesity or for the treatment or prevention of insulin resistance. For example, for a therapeutic agent the assays can be used to determine according to which administration regimen or at which stage of treatment or disease the subject is to be administered the agent. The present invention thus also provides methods for determining whether a subject can be effectively treated with a particular therapy for amelioration of obesity or amelioration or prevention of insulin resistance.

In another aspect, the invention provides a method of predicting a subject's likelihood of developing insulin resistance, comprising:
a) correlating the presence of an allele of the p110β gene with an increased or decreased likelihood of developing insulin resistance and
b) detecting the allele of step a) in the subject, thereby identifying a subject an increased or decreased likelihood of developing insulin resistance.

In a further aspect, described is a method of identifying an allele in the p110β gene correlated with an increased or decreased likelihood of developing insulin resistance, comprising:
a) determining in a subject the presence of an allele of the p110β gene; and
b) correlating the presence of the allele of step (a) with an increased or decreased likelihood of developing insulin resistance, thereby identifying an allele correlated with an increased or decreased likelihood of developing insulin resistance.

Preferably, the methods of the invention comprise determining in the subject the presence or absence of a p110β allele comprising a deletion, insertion or subsitution of one or more nucleic acids in a coding or non-coding region. In a particlarly preferred embodiment, the invention comprises determining in the subject the presence or absence of a p110β allele comprising a deletion, insertion or subsitution of one or more nucleic acids in a regulatory region of the p110β gene, and most preferably a 5′ regulatory region of the p110β gene.

In one aspect, the invention provides methods to determine the course of treatment for an individual, particularly an obese individual. A determination that an individual has an increased susceptibility to insulin resistance indicates that preventative treatment for obesity or insulin-resistance may be beneficial. In another example, a determination that an individual has an increased susceptibility to insulin resistance indicates that aggressive treatment for obesity or insulin resistance may be beneficial. The methods may comprise selecting a treatment regimen, for example dosage of a medicament, in accordance with the likelihood of a subject developing insulin resistance.

In another example, methods of the invention are used to determine a suitable medicament to be administered to a subject, preferably an obese subject. For example, according to present methods, a determination that an individual has an increased susceptibility to insulin resistance may involve treating the individual to diminish insulin resistance or to prevent the development or postpose the onset of insulin resistance. Preferably, treating the individual comprises administering a pharmaceutical agent for the treatment or prevention or insulin resistance.

In a further aspect, the methods of the invention are used to determine the amount of a medicament to be administered to a subject. For example, according to present methods, a determination that an individual has an increased susceptibility to insulin resistance may involve administering a higher dose of an agent for the treatment of obesity and/or insulin resistance that would be administered to an otherwise equivalent individual not determined to have increased susceptibility to insulin resistance.

In another aspect, a determination that an individual suffering from a disorder has a decreased susceptibility to insulin resistance may involve treating the individual for the disorder but not administering an agent for the treatment or prevention of insulin resistance. Preferably said disorder is is obesity.

In another example, the methods are used to assess the therapeutic response of subjects in a clinical trial or to select subjects for inclusion in a clinical trial. For instance, the methods of the invention may comprise determining the genotype of a subject at the p110β gene, wherein said genotype places said subject into a subgroup in a clinical trial or in a subgroup for inclusion in or exclusion from a clinical trial.

As discussed, the invention discloses a method for treating an individual, the method comprising:
(a) determining in the subject the presence or absence of an allele of the p110β gene, wherein the allele is correlated with an increased or decreased likelihood of developing insulin resistance; and
(b) based on said determining in step (a), selecting or determining an effective treatment or an effective amount of an agent for the treatment of obesity or for the treatment or prevention of insulin resistance to administer to said subject.

Also provided is a method for treating an individual, the method comprising: (a) determining whether the individual has increased or decreased p110β expression, wherein increased p110β gene expression is associated with a decreased likelihood of developing insulin resistance; and (b) based on said determining in step (a), selecting or determining an effective treatment or an effective amount of an agent for the treatment of obesity or for the treatment or prevention of insulin resistance to administer to said subject.

In a preferred aspect, the invention discloses a method for treating a human subject, said method comprising:
(a) determining whether the subject suffers from obesity, optionally determining whether the subject has a BMI of at least 25, more preferably of at least 30, or at least 35;
(b) detecting whether the DNA of the subject encodes an allele of the p110β gene correlated with an increased or decreased susceptibility to insulin resistance, or determining whether the subject has increased or decreased levels of p110β expression; and,
(c) administering to the subject an effective amount of a treatment capable of ameliorating or preventing insulin resistance in the subject.

Furthermore, the invention also concerns a method for the treatment of a mammalian subject, preferably a human subject, comprising the following steps:
optionally, genotyping the subject at a p110β allele;
identifying a subject having a p110β allele associated with an increased susceptibility to developing insulin resistance, or who has decreased levels of p110β expression;
following up said individual for the appearance of, and optionally the development of, symptoms related to insulin resistance; and
administering an effective amount of a treatment acting against insulin resistance or against symptoms thereof to said individual at an appropriate stage.

Another embodiment of the present invention comprises a method for the treatment of a mammal, preferably a human, comprising the following steps:
optionally, genotyping an individual at a p110β allele;
selecting an individual having a p110β allele associated with an increased susceptibility to developing insulin resistance, or who has decreased levels of p110β expression;
   administering a preventive treatment against insulin resistance to said individual.

In a further embodiment, the present invention concerns a method for the treatment of a mammal, preferably a human, comprising the following steps:
optionally, genotyping an individual at a p110β allele;
selecting an individual having a p110β allele associated with an increased susceptibility to developing insulin resistance, or who has decreased levels of p110β expression;
administering a preventive treatment against insulin resistance to said individual;
following up said individual for the appearance and the development of symptoms related to insulin resistance; and optionally
administering a treatment acting against insulin resistance or against symptoms thereof to said individual at the appropriate stage.

The present invention also concerns a method of treatment comprising the following steps;
selecting an individual whose DNA encodes a p110β allele associated with increased susceptibility to developing insulin resistance, or who has decreased levels of p110β expression; and
administering a treatment effective against insulin resistance or symptoms thereof, or for the prevention of insulin resistance, to said individual.

In another aspect, the invention provides a method of treatment comprising the following steps;
selecting an individual whose DNA encodes a p110β allele associated with increased susceptibility to developing insulin resistance, or who has decreased levels of p110β expression; and
administering a treatment effective against obesity, or for the prevention of obesity, to said individual.

A DNA sample is obtained from the individual to be tested to determine whether the DNA comprises a p110β allele associated with increased suceptbility to developing insulin resistance. The DNA sample is analyzed to determine whether subject is homozygous or heterozygous for the p110β allele. response when compared to p110β individuals.

The methods of the invention may also be useful in assessing and conducting clinical trials of medicaments. The methods accordingly comprise identifying a first population of individuals who have increased susceptibility to developing insulin resistance and a second population of individuals who have decreased susceptibility to developing insulin resistance in comparison to said first population of individuals. This method may be particularly advantageous for testing of medicaments for the prevention or treatment of insulin resistance. For example, because a trial endpoint may be decreased development of insulin resistance in patients not yet showing insulin resistance or slowed progression of development of insulin resistance, the method allows more accurate assessment of therapeutic response by avoiding the inclusion of patients having either high or low susceptibility to insulin resistance. In one embodiment, the medicament may be administered to the subject in a clinical trial if the DNA sample contains an alleles associated with increased susceptibility to developing insulin resistance and/or if the DNA sample lacks an allele associated with decreased susceptibility to developing insulin resistance.

In preferred aspects, said agent is an insulin sensitizer. Preferably the effective amount of an insulin sensitizer medicament is administered following a determination that a subject, particularly an obese subject, has a genotype associated with increased susceptibility to insulin resisitance or has decreased p110β expression.

The invention also concerns a method for the clinical testing of a medicament, the method comprising the following steps.
administering a medicament to a population of individuals; and
from said population, identifying a first subpopulation of individuals whose DNA comprises a particular p110β allele and a second subpopulation of individuals whose DNA does not comprise said p110β allele.

Said method may further comprise: (a) assessing the response to said medicament in said first subpopulation of individuals; and/or (b) assessing the response to said medicament in said second subpopulation of individuals. In a preferred aspect, the method comprises determining whether a subject comprising a p110β allele develops insulin resistance.

Another preferred aspect concerns a method for the clinical testing of a medicament, preferably a medicament capable of ameliorating or preventing obesity and/or insulin resistance in a human subject. The method comprises the following steps:
identifying a first population of individuals whose DNA comprises a p110β allele and a second population of individuals whose DNA does not comprise said p110β allele; and
administering a medicament, preferably a medicament capable of ameliorating or preventing insulin resistance or obesity in a human subject, to individuals of said first and/or said second population of individuals. In one embodiment, the medicament is administered to individuals of said first population but not to individuals of said second population. In one embodiment, the medicament is administered to individuals of said second population but not to individuals of said first population. In another embodiment, the medicament is administered to the individuals of both said first and said second populations.

Thus, using the method of the present invention, drug efficacy can be assessed by taking account of differences in among drug trial subjects that are more likely to develop insulin resistance. If desired, a trial for evaluation of drug efficacy may be conducted in a population comprised substantially of individuals likely to respond favorably to the medicament, or in a population comprised substantially of individuals likely to respond less favorable to the medicament that another population.

Assessing Obesity and Insulin Resistance

Preferably, an individual to be subjected to the diagnostic, prognostic or treatment methods of the invention is an obese individual. Assessing obesity in a subject preferably comprises assessing body fat content. Preferably, assessing obesity comprises determining a subject's body mass index (BMI), expressed as weight (kg)/height (m²). Although a continuous variable, BMI has been categorized based on epidemiologic data to corresponding to the relative risk of developing comorbid conditions. A BMI of less than 25 is considered to be normal, 25 to 29.9 is overweight, and greater than or equal to 30 is obese. Most preferably, the individual in the methods of the invention is an obese individual, preferably an individual having a BMI of at least 25, preferably at least 30, or preferably at least 35. Alternatively or additionally to measuring body fat content which reflects general adiposity, assessment methods may comprise determining adiposity at specific body sites, for example assessing intra-abdominal fat depots. An individual in the methods of the invention may also be any overweight individual at medical risk because of his weight. This includes generally overweight individuals in the presence of comorbidities, an increased waist cirumference (for example, greater than 88 cm for women or 102 cm for men), or a family history of obesity.

Any suitable methods for assessing insulin resistance known in the art can be used. Insulin resistance may also be referred to in terms of insulin sensitivity, wherein decreased insulin sensitivity corresponds to increased insulin resistance. In one aspect, an oral glucose tolerance test (OGTT) can be used to define impaired glucose tolerance and type 2 diabetes. However, in order to separate functions of insulin secretion and insulin resistance it is preferable to use a standardized test that measures insulin resistance. Insulin resistance can be measured using the euglycaemic clamp method or by modeling the intravenous glucose tolerance test (DeFronzo et al, (1979) AM. J. Physiol 237:E214-E223, the disclosure of which is incorporated herein by reference). Simpler and more convenient method to measure insulin resistance include determining plasma glucose, insulin or pro-insulin or split pro-insulin concentration during an oral glucose tolerance test. In one preferred method, fasting insulin concentrations can be measured in order to estimate insulin resistance (Berman (1989) Diabetes 38:1512-1527, the disclosure of which is incorporated herein by reference.

Preferably, ratios of insulin to glucose concentrations are used as a measure of insulin resistance for fasting values. In a preferred method, insulin resistance is estimated by the insulin tolerance test measuring the rate of decline of blood glucose after the injection of a bolus of insulin. For example, insulin resistance is measured by calculating the rate of decline of the log transformed whole blood glucose concentrations from 3 to 15 minutes afer insulin injection, estimated by linear regression. The number is multiplied by −100 to derice the rate constand Kᵢₜₜ, equivalent to the percentage decline in blood glucose per minute given by the formula 69.3/t_{1/2} where t_{1/2} is the half-life of glucose disappearance. (Phillips et al, (1993) Diabetic Med. 11:286-292), the disclosure of which is incorporated herein by reference.

A mathematic model can be used to calculate an insulin sensitivity index (ISI) and its reciprocal, HOMA-IR (Homeostasis model assessment, described in Matthews et al, (1985) Diabetologia 28:412-419, the disclosure of which is incorporated herein by reference).

Treatment of Insulin Resistance

A number of medicaments for the treatment and prevention of insulin resistance are available for use in accordance with the present invention. The most widely used agents are the thiazolidinedione (TZD) insulin-sensitizing agents. TZDs are a class of compounds that improve insulin action in vivo which have also bee introduced as therapeutic agents for the treatment of type 2 diabetes. Since their introduction, it has been discovered that TZDs act as agonists of the peroxisome proliferator-activated receptor γ (PPAR-γ) nuclear receptor. TZD's have been shown to improve insulin sensitivity in human subjects, including also in obese subjects (Nolan et al, (1994) N. Eng. J. Med. 331: 1188-1193). Examples of suitable TZDs include troglitazone (Eli Lilly, Indianapolis, U.S.A.), rosiglitazone, and pioglitazone.

Treatment of Obesity and Insulin Resistance

Obesity can be treated by nonpharmacological, pharmacological and surgical means. A realistic treatment goal is usually loss of 5-10% of initial body weight over a 6-12 month period, followed by long-tem maintenance of reduced weight. Further details are provided found at National Institutes of Health, National Heart, Lung and Blood Institute and North American Association for the Study of Obesity, (2000) The practical guide: identification, evaluation, and treatment of overweight and obesity in adults, NIH, Bethesda, Md., USA, Publication No. 00-4084. Nonpharmacological treatment for obesity include behavior therapy, exercise and calorie-restricted diets. Pharmacological treatments include sibutramine (Meridia, Abbott Laboratories Abbot Park, Illinois USA), which is a reuptake inhibitor of serotonin, norepinephrine, and dopamine initially developed as an antidepressant. Another therapeutic agent is orlistat (Xenical, Hoffman-LaRoche, Nutley, N.J., USA), an inhibitor of gastrointestinal lipases that blocks 30% of dietary fat from being absorbed. Other agents include noradrenergic-releasing agents such as phentermine drugs (Ionamin, Fastin, Adipex) and diethylpropion (Tenuate). Surgical treatments of obesity include the Roux-en-Y gastric bypass, in which a small gastric pouch prevents the patient from eating large quantities at a single meal and a gastrojejunostomy which produces a dumping syndrome upon injestion of high-carbohydrate foods. Other surgical procedures include laparoscopic adjustable-banded gastroplasty.

Detecting p110β

It is contemplated that other mutations in the p110β gene may be identified in accordance with the present invention by detecting a nucleotide change in particular nucleic acids (U.S. Pat. No. 4,988,617, incorporated herein by reference). A variety of different assays are contemplated in this regard, including but not limited to, fluorescent in situ hybridization (FISH; U.S. Pat. No. 5,633,365 and U.S. Pat. No. 5,665,549, each incorporated herein by reference), direct DNA sequencing, PFGE analysis, Southern or Northern blotting, single-stranded conformation analysis (SSCA), RNAse protection assay, allele-specific oligonucleotide (ASO e.g., U.S. Pat. No. 5,639,611), dot blot analysis denaturing gradient gel electrophoresis (e.g., U.S. Pat. No. 5,190,856 incorporated herein by reference). RFLP (e.g., U.S. Pat. No. 5,324,631 incorporated herein by reference) and PCR-SSCP. Methods for detecting and quantitating gene sequences in for example biological fluids are described in U.S. Pat. No. 5,496,699, incorporated herein by reference.

Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

SEQ ID NO 1 provides a genomic DNA sequences comprising part of the 5′ proximal region of the p110β gene, respectively. Any difference in nucleotide sequence between p110β alleles may be used in the methods of the invention in order to detect and distinuguish the particular p110β allele in an individual. The present invention encompasses polynucleotides for use as primers and probes in the methods of the invention. These polynucleotides may consist of, consist essentially of, or comprise a contiguous span of nucleotides of a sequence from any sequence provided herein as well as sequences which are complementary thereto (“complements thereof’). The “contiguous span” may be at least 25, 35, 40, 50, 70, 80, 100, 250, 500 or 1000 nucleotides in length, to the extent that a contiguous span of these lengths is consistent with the lengths of the particular Sequence ID. It should be noted that the polynucleotides of the present invention are not limited to having the exact flanking sequences surrounding a target sequence of interest, which are enumerated in the Sequence Listing. Rather, it will be appreciated that the flanking sequences surrounding the polymorphisms, or any of the primers of probes of the invention which, are more distant from the markers, may be lengthened or shortened to any extent compatible with their intended use and the present invention specifically contemplates such sequences. It will be appreciated that the polynucleotides referred to herein may be of any length compatible with their intended use. Also the flanking regions outside of the contiguous span need not be homologous to native flanking sequences which actually occur in human subjects. The addition of any nucleotide sequence, which is compatible with the nucleotides intended use is specifically contemplated. Preferred polynucleotides may consist of, consist essentially of, or comprise a contiguous span of nucleotides of a sequence from SEQ ID No 1 or 2 as well as sequences which are complementary thereto. The “contiguous span” may be at least 8, 10, 12, 15, 50, 70, 80, 100, 250, 500 or 1000 nucleotides in length.

The probes of the present invention may be designed from the disclosed sequences for any method known in the art, particularly methods which allow for testing if a particular sequence or marker disclosed herein is present. A preferred set of probes may be designed for use in the hybridization assays of the invention in any manner known in the art such that they selectively bind to one allele of a polymorphism, but not the other under any particular set of assay conditions.

Any of the polynucleotides of the present invention can be labeled, if desired, by incorporating a label detectable by spectroscopic, photochemical, biochemical, immunochemical, or chemical means. For example, useful labels include radioactive substances, fluorescent dyes or biotin. Preferably, polynucleotides are labeled at their 3′ and 5′ ends. A label can also be used to capture the primer, so as to facilitate the immobilization of either the primer or a primer extension product, such as amplified DNA, on a solid support. A capture label is attached to the primers or probes and can be a specific binding member which forms a binding pair with the solid phase reagent's specific binding member (e.g. biotin and streptavidin). Therefore depending upon the type of label carried by a polynucleotide or a probe, it may be employed to capture or to detect the target DNA. Further, it will be understood that the polynucleotides, primers or probes provided herein, may, themselves, serve as the capture label. For example, in the case where a solid phase reagent's binding member is a nucleic acid sequence, it may be selected such that it binds a complementary portion of a primer or probe to thereby immobilize the primer or probe to the solid phase. In cases where a polynucleotide probe itself serves as the binding member, those skilled in the art will recognize that the probe will contain a sequence or “tail” that is not complementary to the target. In the case where a polynucleotide primer itself serves as the capture label, at least a portion of the primer will be free to hybridize with a nucleic acid on a solid phase. DNA Labeling techniques are well known to the skilled technician.

Any of the polynucleotides, primers and probes of the present invention can be conveniently immobilized on a solid support. Solid supports are known to those skilled in the art and include the walls of wells of a reaction tray, test tubes, polystyrene beads, magnetic beads, nitrocellulose strips, membranes, microparticles such as latex particles, sheep (or other animal) red blood cells, duracytes) and others. The solid support is not critical and can be selected by one skilled in the art. Thus, latex particles, microparticles, magnetic or non-magnetic beads, membranes, plastic tubes, walls of microtiter wells, glass or silicon chips, sheep (or other suitable animal's) red blood cells and duracytes are all suitable examples. Suitable methods for immobilizing nucleic acids on solid phases include ionic, hydrophobic, covalent interactions and the like. A solid support, as used herein, refers to any material which is insoluble, or can be made insoluble by a subsequent reaction. The solid support can be chosen for its intrinsic ability to attract and immobilize the capture reagent. Alternatively, the solid phase can retain an additional receptor which has the ability to attract and immobilize the capture reagent. The additional receptor can include a charged substance that is oppositely charged with respect to the capture reagent itself or to a charged substance conjugated to the capture reagent. As yet another alternative, the receptor molecule can be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid phase thus can be a plastic, derivatized plastic, magnetic or non-magnetic metal, glass or silicon surface of a test tube, microtiter well, sheet, bead, microparticle, chip, sheep (or other suitable animal's) red blood cells, duracytes and other configurations known to those of ordinary skill in the art. The polynucleotides of the invention can be attached to or immobilized on a solid support individually or in groups of at least 2, 5, 8, 10, 12, 15, 20, or 25 distinct polynucleotides of the inventions to a single solid support. In addition, polynucleotides other than those of the invention may be attached to the same solid support as one or more polynucleotides of the invention.

Any polynucleotide provided herein may be attached in overlapping areas or at random locations on the solid support. Alternatively the polynucleotides of the invention may be attached in an ordered array wherein each polynucleotide is attached to a distinct region of the solid support which does not overlap with the attachment site of any other polynucleotide. Preferably, such an ordered array of polynucleotides is designed to be “addressable” where the distinct locations are recorded and can be accessed as part of an assay procedure. Addressable polynucleotide arrays typically comprise a plurality of different oligonucleotide probes that are coupled to a surface of a substrate in different known locations. The knowledge of the precise location of each polynucleotides location makes these “addressable” arrays particularly useful in hybridization assays. Any addressable array technology known in the art can be employed with the polynucleotides of the invention. One particular embodiment of these polynucleotide arrays is known as the Genechips, and has been generally described in U.S. Pat. No. 5,143,854; PCT publications WO 90/15070 and 92/10092. These arrays may generally be produced using mechanical synthesis methods or light directed synthesis methods, which incorporate a combination of photolithographic methods and solid phase oligonucleotide synthesis (Fodor et al., Science, 251: 767-777, 1991). The immobilization of arrays of oligonucleotides on solid supports has been rendered possible by the development of a technology generally identified as “Very Large Scale Immobilized Polymer Synthesis” (VLSIPS) in which, typically, probes are immobilized in a high density array on a solid surface of a chip. Examples of VLSIPS technologies are provided in U.S. Pat. Nos. 5,143,854 and 5,412,087 and in PCT Publications WO 90/15070, WO 92/10092 and WO 95/11995, which describe methods for forming oligonucleotide arrays through techniques such as light directed synthesis techniques. In designing strategies aimed at providing arrays of nucleotides immobilized on solid supports, further presentation strategies were developed to order and display the oligonucleotide arrays on the chips in an attempt to maximize hybridization patterns and sequence information. Examples of such presentation strategies are disclosed in PCT Publications WO 94/12305, WO 94/11530, WO 97/29212 and WO 97/31256.

Template Dependent Amplification Methods

A number of template dependent processes are available to amplify the marker sequences present in a given template sample. One of the best known amplification methods is the polymerase chain reaction (referred to as PCR) which is described in detail in U.S. Pat. Nos. 4,683,195, 4,683,202 and 4,800,159, and in Innis et al., PCR Protocols, Academic Press, Inc. San Diego Calif., 1990., each of which is incorporated herein by reference in its entirety.

Briefly, in PCR, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR amplification procedure may be performed in order to quantify the amount of mRNA amplified. Methods of reverse transcribing RNA into cDNA are well known and described in Sambrook et al., In: Molecular Cloning. A Laboratory Manual. 2d Ed., Cold Spring Harbor Laboratory Press, Cold Spring Harbor, N.Y., 1989. Alternative methods for reverse transcription utilize thermostable, RNA-dependent DNA polymerases. These methods are described in WO 90/07641. Polymerase chain reaction methodologies are well known in the art.

Another method for amplification is the ligase chain reaction (“LCR” U.S. Pat. Nos. 5,494,810, 5,484,699, EPO No. 320 308, each incorporated herein by reference). In LCR, two complementary probe pairs are prepared, and in the presence of the target sequence, each pair will bind to opposite complementary strands of the target such that they abut. In the presence of a ligase, the two probe pairs will link to form a single unit.

By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, an RNA-directed RNA polymerase, can be used as yet another amplification method in the present invention. In this method, a replicative sequence of RNA that has a region complementary to that of a target is added to a sample in the presence of an RNA polymerase. The polymerase will copy the replicative sequence that can then be detected. Similar methods also are described in U.S. Pat. No. 4,786,600, incorporated herein by reference, which concerns recombinant RNA molecules capable of serving as a template for the synthesis of complementary single-stranded molecules by RNA-directed RNA polymerase. The product molecules so formed also are capable of serving as a template for the synthesis of additional copies of the original recombinant RNA molecule.

An isothermal amplification method, in which restriction endonucleases and ligases are used to achieve the amplification of target molecules that contain nucleotide 5′-[alpha-thio]-triphosphates in one strand of a restriction site also may be useful in the amplification of nucleic acids in the present invention (Walker et al, (1992), Proc. Nat'l Acad. Sci. USA, 89:392-396; U.S. Pat. No. 5,270,184 incorporated herein by reference). U.S. Pat. No. 5,747,255 (incorporated herein by reference) describes an isothermal amplification using cleavable oligonucleotides for polynucleotide detection. In the method described therein, separated populations of oligonucleotides are provided that contain complementary sequences to one another and that contain at least one scissile linkage which is cleaved whenever a perfectly matched duplex is formed containing the linkage. When a target polynucleotide contacts a first oligonucleotide cleavage occurs and a first fragment is produced which can hybridize with a second oligonucleotide. Upon such hybridization, the second oligonucleotide is cleaved releasing a second fragment that can in turn, hybridize with a first oligonucleotide in a manner similar to that of the target polynucleotide.

Strand Displacement Amplification (SDA) is another method of carrying out isothermal amplification of nucleic acids which involves multiple rounds of strand displacement and synthesis, i.e., nick translation (e.g., U.S. Pat. Nos. 5,744,311; 5,733,752; 5,733,733; 5,712,124). A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization. the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present invention. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (Kwok et al., (1989) Proc. Nat'l Acad. Sci. USA, 86:1173; and WO 88/10315, incorporated herein by reference in their entirety). In NASBA, the nucleic acids can be prepared for amplification by standard phenyl/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNA's are reverse transcribed into single stranded DNA, which is then converted to double stranded DNA, and then transcribed once again with an RNA polymerase such as T7 or SP6. The resulting products. whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA; and double-stranded DNA (dsDNA), which may be used in accordance with the present invention. The ssRNA is a template for a first primer oligonucleotide, which is elongated by reverse transcriptase (RNA-dependent DNA polymerase). The RNA is then removed from the resulting DNA:RNA duplex by the action of ribonuclease H(RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes. this amplification can be done isothermally without addition of enzymes at each cycle. Because of the cyclical nature of this process, the starting sequence can be chosen to be in the form of either DNA or RNA.

PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, In: PCR Protocols. A Guide To Methods And Applications, Academic Press, N.Y., 1990.; and O'hara et al., (1989) Proc. Nat'l Acad. Sci. USA, 86: 5673-5677; each herein incorporated by reference in their entireties).

Methods based on ligation of two (or more) oligonucleotides in the presence of nucleic acid having the sequence of the resulting “di-oligonucleotide”, thereby amplifying the di-oligonucleotide, also may be used in the amplification step of the present, invention. (Wu et al., (1989) Genomics, 4:560, incorporated herein by reference).

Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder. Physical Biochemistry Applications to Biochemistry and Molecular Biology, 2nd ed. Wm. Freeman and Co., New York, N.Y., 1982.

Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al., 1989. For example, chromophore or radiolabel probes or primers identify the target during or following amplification.

One example of the foregoing is described in U.S. Pat. No. 5,279,721, incorporated herein by reference, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, (1994) Hum. Mutat., 3:126-132, 1994). The present invention provides methods by which any or all of these types of analyses may be used. Using the sequences disclosed herein, oligonucleotide primers may be designed to permit the amplification of sequences throughout the p110β gene that may then be analyzed by direct sequencing.

Any of a variety of sequencing reactions known in the art can be used to directly sequence the p110β gene by comparing the sequence of the sample with the corresponding wild-type (control) sequence. Examples of sequencing reactions include those based on techniques developed by Maxam and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays.

Kit Components

All the essential materials and reagents required for detecting and sequencing p110β and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

Design and Titeoretical Considerations for Relative Quantitative RT-PCR™

Reverse transcription (RT) of RNA to cDNA followed by relative quantitative PCR (RT-PCR) can be used to determine the relative concentrations of specific mRNA species isolated from subjects. Quantitative PCR may be useful for example in examining relative levels of p110β and p110β mRNA in subjects. Since increased expression of p110β is associated with decreased susceptibility to insulin resistance, this method may be used to determine whether a subject has increased or decreased susceptibility to insulin resistance.

In PCR, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

The second condition that must be met for an RT-PCR experiment to successfully determine the relative abundances of a particular mRNA species is that relative concentrations of the amplifiable cDNAs must be normalized to some independent standard. The goal of an RT-PCR experiment is to determine the abundance of a particular mRNA species relative to the average abundance of all mRNA species in the sample. In the experiments described below, mRNAs for p110β can be used as standards to which the relative abundance of p110β mRNAs are compared.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an RT-PCR assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR assay with an external standard protocol. These assays sample the PCR products in the linear portion of their amplification curves. The number of PCR cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR assay with an internal standard.

One reason for this advantage is that without the internal standard/competitor, all of the reagents can be converted into a single PCR product in the linear range of the amplification curve, thus increasing the sensitivity of the assay. Another reason is that with only one PCR product, display of the product on an electrophoretic gel or another display method becomes less complex, has less background and is easier to interpret.

Chip Technologies

Specifically contemplated by the present inventors are chip-based DNA technologies such as those described by Hacia et al., ((1996) Nature Genetics, 14:441-447) and Shoemaker et al., ((1996) Nature Genetics 14:450-456. Briefly, these techniques involve quantitative methods for analyzing large numbers of genes rapidly and accurately. By tagging genes with oligonucleotides or using fixed probe arrays, one can employ chip technology to segregate target molecules as high density arrays and screen these molecules on the basis of hybridization. See also Pease et al, ((1994) Proc. Nat'l Acad. Sci. USA, 91:5022-5026); Fodor et al., ((1991) Science, 251:767-773).

Methods of Detecting p110β Protein

Antibodies can be used in characterizing the p110β content of tissues, through techniques such as ELISAs and Western blotting.

In one example, p110β antibodies can be used in an ELISA assay is contemplated. For example, anti-p110β antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for p110β that differs the first antibody. Appropriate conditions preferably include diluting the sample with diluents such as BSA, bovine gamma globulin (BGG) and phosphate buffered saline (PBS)/Tween. These added agents also tend to assist in the reduction of nonspecific background. The layered antisera is then allowed to incubate for from about 2 to about 4 hr, at temperatures preferably on the order of about 25 C to about 27 C. Following incubation, the antisera-contacted surface is washed so as to remove non-immunocomplexed material. A preferred washing procedure includes washing with a solution such as PBS/Tween or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 hr at room temperature in a PBS-containing solution such as PBS/Tween).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The steps of various other useful immunodetection methods have been described in the scientific literature, such as, eg., Nakamura et al., In: Handbook of Experimental Immunology (4th Ed.), Weir. E., Herzenberg, L. A. Blackwell, C., Herzenberg, L. (eds). Vol. 1. Chapter 27, Blackwell Scientific Publ., Oxford, 1987; incorporated herein by reference). Immunoassays, in their most simple and direct sense, are binding assays. Certain preferred immunoassays are the various types of radioimmunoassays (RIA) and immunobead capture assay. Immunohistochemical detection using tissue-sections also is particularly useful. However, it will be readily appreciated that detection is not limited to such techniques, and Western blotting, dot blotting, FACS analyses, and the like also may be used in connection with the present invention.

Antibodies specific for p110β for use according to the present invention can be obtained using known methods. An isolated p110β protein, or a portion or fragment thereof, can be used as an immunogen to generate antibodies that bind p110β using standard techniques for polyclonal and monoclonal antibody preparation. A p110β protein can be used or, alternatively, the invention provides antigenic peptide fragments of p110β for use as immunogens.

p110β polypeptides can be prepared using known means, either by purification from a biological sample obtained from an individual or more preferably as recombinant polypeptides. The p110β amino acid sequence is shown in SEQ ID NO: 3. The antigenic peptide of p110β preferably comprises at least 8 amino acid residues of the amino acid sequence shown in SEQ ID NO: 3. Said antigenic peptide encompasses an epitope of p110β such that an antibody raised against the peptide forms a specific immune complex with p110β. Preferably, the antigenic peptide comprises at least 10 amino acid residues, more preferably at least 15 amino acid residues, even more preferably at least 20 amino acid residues, and most preferably at least 30 amino acid residues. Preferred epitopes encompassed by the antigenic peptide are regions of p110β that are located on the surface of the protein, e.g., hydrophilic regions.

A p110β immunogen typically is used to prepare antibodies by immunizing a suitable subject, (e.g., rabbit, goat, mouse or other mammal) with the immunogen. An appropriate immunogenic preparation can contain, for example, recombinantly expressed p110β protein or a chemically synthesized p110β polypeptide. The preparation can further include an adjuvant, such as Freund's complete or incomplete adjuvant, or similar immunostimulatory agent. Immunization of a suitable subject with an immunogenic p110β preparation induces a polyclonal anti-p110β antibody response.

Accordingly, another aspect of the invention pertains to anti-p110β antibodies. The term “antibody” as used herein refers to immunoglobulin molecules and immunologically active portions of immunoglobulin molecules, i.e., molecules that contain an antigen binding site which specifically binds (immunoreacts with) an antigen, such as p110β. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind p110β. The term “monoclonal antibody” or “monoclonal antibody composition”, as used herein, refers to a population of antibody molecules that contain only one species of an antigen binding site capable of immunoreacting with a particular epitope of p110β. A monoclonal antibody composition thus typically displays a single binding affinity for a particular p110β protein with which it immunoreacts.

Polyclonal anti-p110β antibodies can be prepared as described above by immunizing a suitable subject with a p110β immunogen. The anti-p110β antibody titer in the immunized subject can be monitored over time by standard techniques, such as with an enzyme linked immunosorbent assay (ELISA) using immobilized p110β. If desired, the antibody molecules directed against p110β can be isolated from the mammal (e.g., from the blood) and further purified by well known techniques, such as protein A chromatography to obtain the IgG fraction. At an appropriate time after immunization, e.g., when the anti-p110β antibody titers are highest, antibody-producing cells can be obtained from the subject and used to prepare monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) PNAS 76:2927-31; and Yeh et al. (1982) Int. J. Cancer 29:269-75), the more recent human B cell hybridoma technique (Kozbor et al. (1983) Immunol Today 4:72), the EBV-hybridoma technique (Cole et al. (1985), Monoclonal Antibodies and Cancer Therapy, Alan R. Liss, Inc., pp. 77-96) or trioma techniques. The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lemer (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a p110β immunogen as described above, and the culture supernatants of the resulting hybridoma cells are screened to identify a hybridoma producing a monoclonal antibody that binds p110β.

Any of the many well known protocols used for fusing lymphocytes and immortalized cell lines can be applied for the purpose of generating an anti-p110β monoclonal antibody (see, e.g., G. Galfre et al. (1977) Nature 266:55052; Gefter et al. Somatic Cell Genet., cited supra; Lemer, Yale J. Biol. Med, cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind p110β, e.g., using a standard ELISA assay.

As an alternative to preparing monoclonal antibody-secreting hybridomas, a monoclonal anti-p110β antibody can be identified and isolated by screening a recombinant combinatorial immunoglobulin library (e.g., an antibody phage display library) with p110β to thereby isolate immunoglobulin library members that bind p110β. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, Ladner et al. U.S. Pat. No. 5,223,409; Kang et al. PCT International Publication No. WO 92/18619; Dower et al. PCT International Publication No. WO 91/17271; Winter et al. PCT International Publication WO 92/20791; Markland et al. PCT International Publication No. WO 92/15679; Breitling et al. PCT International Publication WO 93/01288; McCafferty et al. PCT International Publication No. WO 92/01047; Garrard et al. PCT International Publication No. WO 92/09690; Ladner et al. PCT International Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734; Hawkins et al. (1992) J. Mol. Biol. 226:889-896; Clarkson et al. (1991) Nature 352:624-628; Gram et al. (1992) PNAS 89:3576-3580; Garrad et al. (1991) Bio/Technology 9:1373-1377; Hoogenboom et al. (1991) Nuc. Acid Res. 19:4133-4137; Barbas et al. (1991) PNAS 88:7978-7982; and McCafferty et al. Nature (1990) 348:552-554.

An anti-p110β antibody (e.g., monoclonal antibody) can be used to isolate p110β by standard techniques, such as affinity chromatography or immunoprecipitation. An anti-p110β antibody can facilitate the purification of natural p110β from cells and of recombinantly produced p110β expressed in host cells. Moreover, an anti-p110β antibody can be used to detect p110β protein (e.g., in a cellular lysate or cell supernatant) in order to evaluate the abundance and pattern of expression of the p110β protein. Anti-p110β antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, -galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Method for Screening Ligands that Modulate the Expression of the p110β Gene

The present inventors have shown that an allele in the 5′ upstream regulatory region of the p110β gene results in the modification of p110β expression levels. In particular, it is shown that an allele of the p110β gene associated with decreased likelihood of developing insulin resistance also results in the modification of p110β expression levels Another subject of the present invention is therefore a method for screening molecules that modulate the expression of the p110β protein.

Such a screening method comprises the steps of:
a) cultivating a prokaryotic or an eukaryotic cell that has been transfected with a nucleotide sequence encoding the p110β protein or a variant or a fragment thereof, placed under the control of its own promoter;
b) bringing into contact the cultivated cell with a molecule to be tested;
c) quantifying the expression of the p110β protein or a variant or a fragment thereof.

In one embodiment, the nucleotide sequence encoding the p110β protein or a variant or a fragment thereof comprises an allele of at least one p110β polymorphism in a regulatory region, preferably the polymorphism at nucleotide position 100 of SEQ ID NO 1, or at position −359 (position 359 upstream from the start codon) of the p110β gene.

Using DNA recombination techniques well known by the one skill in the art, the p110β protein encoding DNA sequence is inserted into an expression vector, downstream from its promoter sequence. As an illustrative example, the promoter sequence of the p110β gene is contained in the nucleic acid of the 5′ regulatory region. The quantification of the expression of the p110β protein may be realized either at the mRNA level or at the protein level. In the latter case, polyclonal or monoclonal antibodies may be used to quantify the amounts of the p110β protein that have been produced, for example in an ELISA or a RIA assay. In a preferred embodiment, the quantification of the p110β mRNA is realized by a quantitative PCR amplification of the cDNA obtained by a reverse transcription of the total mRNA of the cultivated p110β -transfected host cell, using a pair of primers specific for p110β.

The present invention also concerns a method for screening substances or molecules that are able to modulate (increase or decrease) the level of expression of the p110β gene. Said substances or molecules may be useful for preventing insulin resistance in a subject or treating a subject susceptible to developing or suffereing from insulin resistance. Said substances or molecules may be particularly useful for treating or preventing insulin resistance in obese subjects. Preferred compounds for treating or preventing insulin resistance are compounds that increase p110β expression. Such methods of screening may allow the one skilled in the art to select substances exerting a regulating effect on the expression level of the p110β gene and which may be useful as active ingredients included in pharmaceutical compositions for treating patients suffering from diseases.

Thus, is also part of the present invention a method for screening of a candidate substance or molecule that modulated the expression of the p110β gene, this method comprises the following steps:
(a) providing a recombinant cell host containing a nucleic acid, wherein said nucleic acid comprises a nucleotide sequence of the 5′ regulatory region of the p110β gene, or a biologically active fragment or variant thereof, located upstream of a polynucleotide encoding a detectable protein;
(b) obtaining a candidate substance; and
(c) determining the ability of the candidate substance to modulate the expression levels of the polynucleotide encoding the detectable protein.

In one embodiment, the nucleotide sequence encoding the p110β protein or a variant or a fragment thereof comprises a contiguous span of at least 15, 20, 30, 50, 75, 100, 200, 300 or 500 nucleotides of SEQ ID NO 1 or 3, or the complement thereof, to the extent that the length of said SEQ ID NO is consistent therewith. In a preferred aspect, the nucleotide sequence encoding the p110β protein or a variant or a fragment thereof is at least 50%, 60%, 70%, 80%, 90%, 95% or 99% identical to a contiguous span of at least 15, 20, 30, 50, 75, 100, 200, 300 or 500 nucleotides of SEQ ID NO 1 or 3, or the complement thereof. Preferably, the nucleotide sequence encoding the p110β protein or a variant or a fragment thereof comprises an allele of at least one p110β polymorphism in the 5′ regulatory region, preferably the polymorphism at nucleotide position 100 of SEQ ID NO 1, or at position −359 (position 359 upstream from the start codon) of the p110β gene.

In a further embodiment, the nucleic acid comprising the nucleotide sequence of the 5′ regulatory region or a biologically active fragment or variant thereof also includes a 5′UTR region of the p110β cDNA of SEQ ID No 2, or one of its biologically active fragments or variants thereof.

Examples of polynucleotides encoding a detectable protein include polynucleotides encoding beta galactosidase, luciferase, green fluorescent protein (GFP) and chloramphenicol acetyl transferase (CAT).

The invention also pertains to kits useful for performing the herein described screening method. Preferably, such kits comprise a recombinant vector that allows the expression of a nucleotide sequence of the 5′ regulatory region or a biologically active fragment or variant thereof located upstream and operably linked to a polynucleotide encoding a detectable protein or the p110β protein or a fragment or a variant thereof.

In another embodiment of a method for the screening of a candidate substance or molecule that modulates the expression of the p110β gene, wherein said method comprises the following steps:
a) providing a recombinant host cell containing a nucleic acid, wherein said nucleic acid comprises a 5′UTR sequence of a p110β cDNA, preferably of a p110β cDNA of SEQ ID No 2, or one of its biologically active fragments or variants, the 5′UTR sequence or its biologically active fragment or variant being operably linked to a polynucleotide encoding a detectable protein;
b) obtaining a candidate substance; and
c) determining the ability of the candidate substance to modulate the expression levels of the polynucleotide encoding the detectable protein.

In a specific embodiment of the above screening method, the nucleic acid that comprises a nucleotide sequence selected from the group consisting of the 5′UTR sequence of a p110β cDNA, preferably of a p110β cDNA of SEQ ID No 2 or one of its biologically active fragments or variants, includes a promoter sequence which is endogenous with respect to the p110 β 5′UTR sequence. In another specific embodiment of the above screening method, the nucleic acid that comprises a nucleotide sequence selected from the group consisting of the 5′UTR sequence of a p110β cDNA or one of its biologically active fragments or variants, includes a promoter sequence which is exogenous with respect to the p110 β 5′UTR sequence defined therein.

The invention further comprises a kit for the screening of a candidate substance modulating the expression of the p110β, gene, wherein said kit comprises a recombinant vector that comprises a nucleic acid from a 5′ reglatory region of the p110β gene and/or a 5′ UTR sequence of the p110β cDNA, or one of their biologically active fragments or variants, the 5′ regulatory region-derived sequence or the UTR sequence or its biologically active fragment or variant being operably linked to a polynucleotide encoding a detectable protein.

For the design of suitable recombinant vectors useful for performing the screening methods described above, it will be referred to the section of the present specification wherein the preferred recombinant vectors of the invention are detailed.

Expression levels and patterns of p110β may be analyzed by solution hybridization with long probes as described in International Patent Application No. WO 97/05277. Briefly, the p110β cDNA or the p110β genomic DNA described above, or fragments thereof, is inserted at a cloning site immediately downstream of a bacteriophage (T3, T7 or SP6) RNA polymerase promoter to produce antisense RNA. Preferably, the p110β insert comprises at least 100 or more consecutive nucleotides of the genomic DNA sequence or the cDNA sequences. The plasmid is linearized and transcribed in the presence of ribonucleotides comprising modified ribonucleotides (i.e. biotin-UTP and DIG-UTP). An excess of this doubly labeled RNA is hybridized in solution with mRNA isolated from cells or tissues of interest. The hybridization is performed under standard stringent conditions (40-50° C. for 16 hours in an 80% formamide, 0.4 M NaCl buffer, pH 7-8). The unhybridized probe is removed by digestion with ribonucleases specific for single-stranded RNA (i.e. RNases CL3, T1, Phy M, U2 or A). The presence of the biotin-UTP modification enables capture of the hybrid on a microtitration plate coated with streptavidin. The presence of the DIG modification enables the hybrid to be detected and quantified by ELISA using an anti-DIG antibody coupled to alkaline phosphatase.

Methods for quantitative analysis of p110β gene expression are further described herein. Quantitative analysis may also be performed using arrays. As used herein, the term array means a one dimensional, two dimensional, or multidimensional arrangement of a plurality of nucleic acids of sufficient length to permit specific detection of expression of mRNAs capable of hybridizing thereto. For example, the arrays may contain a plurality of nucleic acids derived from genes whose expression levels are to be assessed. The arrays may include the p110β genomic DNA, the p110β cDNA sequences or the sequences complementary thereto or fragments thereof, particularly those comprising at least one of the biallelic markers according the present invention. Preferably, the fragments are at least 15 nucleotides in length. In other embodiments, the fragments are at least 25 nucleotides in length. In some embodiments, the fragments are at least 50 nucleotides in length. More preferably, the fragments are at least 100 nucleotides in length. In another preferred embodiment, the fragments are more than 100 nucleotides in length. In some embodiments the fragments may be more than 500 nucleotides in length.

For example, quantitative analysis of p110β gene expression may be performed with a complementary DNA microarray as described by Schena et al (1995) (Science. 270:467-470) and (1996) (Proc. Natl. Acad. Sci. U.S.A. 93(20):10614-10619). Full-length p110β cDNA or fragment thereof is amplified by PCR and arrayed from a 96-well microtiter plate onto silylated microscope slides using high-speed robotics. Printed arrays are incubated in a humid chamber to allow rehydration of the array elements and rinsed, once in 0.2% SDS for 1 min, twice in water for 1 min, and once for 5 min in sodium borohydride solution. The arrays are submerged in water for 2 min at 95° C., transferred into 0.2% SDS for 1 min, rinsed twice with water, air dried and stored in the dark at 25° C.

Cell or tissue mRNA is isolated or commercially obtained and probes are prepared by a single round of reverse transcription. Probes are hybridized to 1 cm² microarrays under a 14×14 mm glass coverslip for 6-12 hours at 60° C. Arrays are washed for 5 min at 25° C. in low stringency wash buffer (1×SSC/0.2% SDS), then for 10 min at room temperature in high stringency wash buffer (0.1×SSC/0.2% SDS). Arrays are scanned in 0.1×SSC using a fluorescence laser scanning device fitted with a custom filter set. Accurate differential expression measurements are obtained by taking the average of the ratios of two independent hybridizations.

Quantitative analysis of p110β gene expression may also be performed with full length p110β cDNAs or fragments thereof in complementary DNA arrays as described by Pietu et al (1996) (Genome Research. 6:492-503). The full-length p110β cDNA or fragments thereof is PCR amplified and spotted on membranes. Then, mRNAs originating from various tissues or cells are labeled with radioactive nucleotides. After hybridization and washing in controlled conditions, the hybridized mRNAs are detected by phospho-imaging or autoradiography. Duplicate experiments are performed and a quantitative analysis of differentially expressed mRNAs is then performed.

Alternatively, expression analysis using the p110β genomic DNA, the p110β cDNA, or fragments thereof can be done through high density nucleotide arrays as described by Lockhart et al (1996) (Nature Biotechnology 14:1675-1680) and Sosnowsky et al (1997) (Proc. Natl. Acad. Sci. U.S.A. 94:1119-1123). Oligonucleotides of 15-50 nucleotides from the sequences of the p110β genomic DNA, the p110β cDNA sequences particularly those comprising at least one of biallelic markers according the present invention, or the sequences complementary thereto, are synthesized directly on the chip (Lockhart et al., supra) or synthesized and then addressed to the chip (Sosnowski et al., supra). Preferably, the oligonucleotides are about 20 nucleotides in length. p110β cDNA probes labeled with an appropriate compound, such as biotin, digoxigenin or fluorescent dye, are synthesized from the appropriate mRNA population and then randomly fragmented to an average size of 50 to 100 nucleotides. The said probes are then hybridized to the chip. After washing as described in Lockhart et al., supra and application of different electric fields (Sosnowsky et al., 1997)., the dyes or labeling compounds are detected and quantified. Duplicate hybridizations are performed. Comparative analysis of the intensity of the signal originating from cDNA probes on the same target oligonucleotide in different cDNA samples indicates a differential expression of p110β mRNA.

Method for Screening Substances Interacting with the Regulatory Sequences of a p110β Gene

The present invention also concerns a method for screening substances or molecules that are able to interact with the regulatory sequences of the p110β gene, such as for example promoter or enhancer sequences.

Nucleic acids encoding proteins which are able to interact with the regulatory sequences of the p110β gene, more particularly a nucleotide sequence selected from the group consisting of the polynucleotides of the 5′ and 3′ regulatory region or a fragment or variant thereof, and more preferably a nucleotide sequence comprising the polymorphic base at position 100 of SEQ ID NO 1 or at position −359 upstream of the start codon of the p110β gene of the invention, may be identified by using a one-hybrid system, such as that described in the booklet enclosed in the Matchmaker One-Hybrid System kit from Clontech (Catalog Ref. no K1603-1), the technical teachings of which are herein incorporated by reference. Briefly, the target nucleotide sequence is cloned upstream of a selectable reporter sequence and the resulting DNA construct is integrated in the yeast genome (Saccharomyces cerevisiae). The yeast cells containing the reporter sequence in their genome are then transformed with a library comprising fusion molecules between cDNAs encoding candidate proteins for binding onto the regulatory sequences of the p110β gene and sequences encoding the activator domain of a yeast transcription factor such as GAL4. The recombinant yeast cells are plated in a culture broth for selecting cells expressing the reporter sequence. The recombinant yeast cells thus selected contain a fusion protein that is able to bind onto the target regulatory sequence of the p110β gene. Then, the cDNAs encoding the fusion proteins are sequenced and may be cloned into expression or transcription vectors in vitro. The binding of the encoded polypeptides to the target regulatory sequences of the p110β gene may be confirmed by techniques familiar to the one skilled in the art, such as gel retardation assays or DNAse protection assays.

Gel retardation assays may also be performed independently in order to screen candidate molecules that are able to interact with the regulatory sequences of the p110β gene, such as described by Fried and Crothers (1981) (Nucleic Acids Res. 9:6505-6525), Garner and Revzin (1981) (Nucleic Acids Res. 9:3047-3060) and Dent and Latchman (1993) (The DNA mobility shift assay. In: Transcription Factors: A Practical Approach (Latchman D S, ed.) Oxford: IRL Press. pp 1-26), the teachings of these publications being herein incorporated by reference. These techniques are based on the principle according to which a DNA fragment which is bound to a protein migrates slower than the same unbound DNA fragment. Briefly, the target nucleotide sequence is labeled. Then the labeled target nucleotide sequence is brought into contact with either a total nuclear extract from cells containing transcription factors, or with different candidate molecules to be tested. The interaction between the target regulatory sequence of the p110β gene and the candidate molecule or the transcription factor is detected after gel or capillary electrophoresis through a retardation in the migration.

The invention will be more fully understood by reference to the following examples. They should not, however, be construed as limiting the scope of the invention. All literature and patent citations are expressly incorporated herein by reference.

### EXAMPLES

### Example 1

PCR Genotyping at p110β Polymorphisms

PCR Genotpying

PCR amplification was carried out in 96-well microtiter plates (Perkin), each 50 μl reaction containing 200 ng DNA, 1.5 mM MgCl2, 5 μl 10× Fast Start Buffer (Roche), 0.2 mM each dNTP, 1 μM of each primer and 1.25 U of Fast Start Taq Polymerase (Roche). 35 cycles were performed using a 9700 Perkin Elmer thermocycler, with an annealing temperature of 47° C.

Primers sequences used for genotyping were:

| | |
|---|---|
| p110-359L: | |
| 5′-CCTGTCAAGTGCTGGTTAACTA-3′ | (SEQ ID NO 5) |
| p110-359U: | |
| 5′-CAATCCATACCACCAACTAAAG-3′ | (SEQ ID NO 6) |

10 μl of PCR product were digested with 1 U of Ssp1 (10 U/μl) (Invitrogen). Each digested product was detected by ethidium bromide staining after electrophoresis in a 2% agarose gel in 0.5×TBE.

Patients having TT genotype at the −359 position of the p110β gene showed bands of 116 and 75 bp. Patients having TC genotype showed bands of 191, 116 and 75 bp. Patients having the CC genotype had a band of 191 bp.

### Example 2

Association of p110β with Insulin Resistance in Obese Children

Two cohorts of children suffering from obesity were examined for association of the polymorphism at position −359 (359 nucleotides upstream of the start codon) of the p110β gene with insulin response. Either a T or a C is commonly present at position −359 of the p110β gene.

In a first cohort of 358 children, 84 patients were found to have TT genotype, 179 had TC genotype, and 95 patients were CC homozygotes. In a second cohort of 397 children, 105 patients were found to have TT genotypes, 187 had TC genotypes and 105 patients were CC homozygotes. A summary of the cohorts is shown in Table 1.

Genotyping was carried out on samples as described in Example 1. An insulin resistance index was based on individuals' HOMA IR, as determined using standards methods, as described in Phillips et al. (1993) Diabetic Med. 11: 286-292.

The averge BMI and insulin resistance measures (HOMA IR and plasma fasting insulin) for the (a) TT and TC and (b) CC groups are shown in Table 1 for each of the cohorts. A regression model takes into account BMI and other test factors on HOMA IR. Patients having higher BMI generally have higher insulin resistance. However, when adjusted for BMI individually for each group, patients in the CC group had lower insulin resistance values than patients in the TT and TC groups, particularly at higher BMIs. More particularly, when adjusting for individual BMI within each group, the magnitude of the adjustment was smaller for the CC group than for the other groups. The genotyping group (TT, TC or CC) in the regression model is a categorized classification with 100% certainty. The genotyping, in other embodiments, could be performed in the natural order of increased expression among the three categories, or the actual percentage of the gene expression could be used. Results are shown in FIG. 5 for the first cohort (upper row) and second cohort (lower row). Thus, patients homozygous for the C allele appear to be protected from developing insulin resistance compared to TT and TC patients.

TABLE 1
Means by PI3K genotype for both cohorts

| | Cohort 1 | | | Cohort 2 | | |
|---|---|---|---|---|---|---|
| | TT | CT | CC | TT | CT | CC |
| Individuals | 84 | 179 | 95 | 105 | 187 | 105 |
| % female | 57.1 | 59.8 | 57.9 | 68.6 | 59.4 | 61 |
| Age (y) | 11 ± 0.3 | 12.3 ± 0.2 | 12.3 ± 0.3** | 12.5 ± 0.2 | 11.8 ± 0.2 | 12.5 ± 0.2* |
| BMI (kg/m2) | 29.4 ± 0.7 | 31.9 ± 0.5 | 31.7 ± 0.7* | 32.3 ± 0.6 | 30 ± 0.4 | 32.9 ± 0.7** |
| BMI (% N) | 171.8 ± 2.9 | 176.3 ± 2 | 175 ± 2.9 | 174.6 ± 2.8 | 168 ± 1.9 | 176.8 ± 3.2* |
| Fasting insulin (μU/ml) | 19 ± 1.4 | 19.7 ± 0.9 | 17 ± 0.7 | 22.5 ± 1.6 | 17.6 ± 0.8 | 16.5 ± 0.6** |
| Fasting glucose (mM) | 4.45 ± 0.1 | 4.44 ± 0 | 4.32 ± 0.04 | 4.9 ± 0.1 | 4.7 ± 0 | 4.8 ± 0.05 |
| HOMA IR | 3.9 ± 0.3 | 4.0 ± 0.2 | 3.3 ± 0.15 | 5 ± 0.4 | 3.7 ± 0.2 | 3.5 + 0.1** |

*The means reported are not transformed. However, HOMA and fasting insulin were log transformed to approach normality for the analysis of variance. ANOVA comparisons are unadjusted for other factors.
*p < 0.05
**p < 0.001

TABLE 2
General linear model for regression of BMI and test factors on HOMA
IR, the index reflecting insulin resistance.

| | | | |
|---|---|---|---|
| | | | type III |
| Factor | Estimate (mean ± s.e.) | CI | LRT P value |

Cohort 1 (N = 358)

| | | | | | |
|---|---|---|---|---|---|
| Intercept | | 0,0645 ± 0,123 | (−0,1772 | 0,3063) | |
| BMI | | 0,010 ± 0,003 | (0,0037 | 0,0166) | <.0001 |
| Sex | f | 0,0048 ± 0,0242 | (−0,0521 | 0,0426) | 0,8439 |
| | m | | | | |
| P110 G | TT | −0,5822 ± 0,147 | (−0,8704 | −0,2941) | 0,0001 |
| | CT | −0,4082 ± 0,1209 | (−0,6451 | −0,1712) | |
| | TT | | | | |
| BMI*P110 G | TT | 0,0217 ± 0,0047 | (0,0124 | 0,031) | <.0001 |
| | CT | 0,0144 ± 0,0037 | (0,0071 | 0,0217) | |
| | CC | | | | |
| Age | | 0,0125 ± 0,0105 | (−0,008 | 0,033) | 0,2338 |
| Pub | | −0,0248 ± 0,0197 | (−0,0634 | 0,0137) | 0,207 |
| Scale | | 0,2056 ± 0,0077 | (0,1911 | 0,2212) | |
| Cohort 2 (N = 397) | | | | | |
| Intercept | | −0,2439 ± 0,1045 | (−0,4486 | −0,0391) | |
| BMI | | 0,016 ± 0,0028 | (0,0105 | 0,0215) | <.0001 |
| Sex | f | 0,0678 ± 0,0221 | (0,0244 | 0,1112) | 0,0023 |
| | m | | | | |
| P110 G | TT | −0,4429 ± 0,1359 | (−0,7093 | −0,1765) | 0,0005 |
| | CT | −0,4375 ± 0,122 | (−0,6767 | −0,1983) | |
| | CC | | | | |
| BMI*P110 G | TT | 0,0172 ± 0,0041 | (0,0091 | 0,0252) | <.0001 |
| | CT | 0,0157 ± 0,0038 | (0,0082 | 0,0231) | |
| | CC | | | | |
| Age | | 0,0205 ± 0,0073 | (0,0062 | 0,0347) | 0,0051 |
| Pub | | −0,0281 ± 0,0136 | (−0,0548 | −0,0013) | 0,0402 |
| Scale | | 0,1936 ± 0,0069 | (0,1806 | 0,2075) | |

### Example 3

p110β Expression Assays

p110β Promoter of the Phosphatidylinositol 3-kinase Reporter Constructs Fragments of the human p110β promoter containing −359T or −359C were amplified from genomic DNA by using a sense primer (5′-cggggtaccAATCACCTGTCAAGTGCTGGTTAACTAAATTTC-3′) (SEQ ID NO 7) and an antisense primer (5′-gaagatctGGCATTATGAAACTGAAGCACTTACATAACCA-3′) (SEQ ID NO 8). These primers add the KpnI and BglII restriction sites, respectively. The fragment was then inserted upstream from the firefly luciferase-coding sequence in the pGL3 vector plasmid.

Transient Transfections and Luciferase Assays.

NIH-3T3 (mouse embryonic fibroblast cell) cells were obtained from American Type Tissue Culture Collection ATCC). Cells were grown in Dulbecco's modified Eagle's medium supplemented with 10% fetal bovine serum at 37° C. with 5% CO₂. Cells were transfected with plasmids using the FuGENE 6 transfection reagent (Roche Diagnostics). Transfection with FuGENE 6 was carried out according to the instructions of the manufacturer (2×105 cells/6-cm well; 1 μg of reporter gene plasmid, 100 ng of pRL-TK vector for normalization of transfection efficiency, 6 μl of FuGENE 6 transfection reagent). Twenty four hours after transfection, the cells were harvested and luciferase activities were determined using the dual-luciferase reporter assay system as indicated by the manufacturer (Promega).

1. A method of predicting a subject's likelihood of developing insulin resistance, comprising determining in a subject the identity of the nucleotide present at position 100 of SEQ ID NO 1, wherein the allele comprising said nucleotide is correlated with an increased or decreased likelihood of developing insulin resistance, thereby identifying the subject as having an increased or decreased likelihood of developing insulin resistance.

2. The method of claim 1, further comprising administering an effective amount of an agent for the treatment or prevention of insulin resistance to said subject if the subject is identified as having an increased likelihood of developing insulin resistance.

3. A method of predicting a subject's likelihood of developing insulin resistance, comprising determining whether the subject has an increased or decreased level of p110β expression, wherein increased or decreased level of p110β expression is correlated with an increased or decreased likelihood of developing insulin resistance, thereby identifying the subject as having an increased or decreased likelihood of developing insulin resistance.

4. The method of claim 2, further comprising administering an effective amount of an agent for the treatment or prevention of insulin resistance to said subject if the subject is identified as having an increased likelihood of developing insulin resistance.

5. A method for treating or preventing insulin resistance in a subject, the method comprising:
(a) determining the identity of the nucleotide present at position 100 of SEQ ID NO 1, wherein the allele comprising said nucleotide is correlated with an increased or decreased likelihood of developing insulin resistance; and
(b) selecting or determining an effective amount of an agent for the treatment or prevention of insulin resistance to administer to said subject.

6. The method of claim 5 further comprising (c) administering said effective mount of said agent to said subject.

7. A method for treating or preventing insulin resistance in a subject, the method comprising:
(a) determining whether the subject has an increased or decreased level of p110β expression, wherein an increased or decreased level of p110β expression is correlated with an increased or decreased likelihood of developing insulin resistance; and
(b) determining an effective amount of an agent for the treatment or prevention of insulin resistance to administer to said subject.

8. The method of claim 7 further comprising (c) administering said effective amount of said agent to said subject.

9. A method of identifying a test compound that modulates p110β transcription, said method comprising:
comparing the level of transcription from a p110β regulatory region in the presence and absence of a test compound wherein a determination that the level of transcription is increased or decreased in the presence of said test compound relative to the level of transcription in the absence of said test compound indicates that said compound is a candidate modulator of transcription.

10. A method of identifying a test compound for the treatment or prevention of insulin resistance according to claim 1, wherein a determination that the level of transcription is increased or decreased in the presence of said test compound relative to the level of transcription in the absence of said test compound indicates that said compound is a candidate compound for the treatment or prevention of insulin resistance.

11. The method of claim 9, wherein the level of transcription from said p110β regulatory region in the presence and absence of the test compound is determined by performing an in vitro transcription reaction using a construct comprising said p110β regulatory region.

12. The method of claim 9, wherein the level of transcription from said p110β responsive regulatory region in the presence and the absence of the test compound is determined by measuring the level of transcription from a p110β regulatory region in a cell.

13. The method of claim 9, wherein said p110β regulatory region comprises a contiguous span of at least 30 nucleotides of SEQ ID No 1 or the complements thereof, or homologs thereof having at least 60% nucleotide identity.

14. The method of claim 9, wherein said p110β regulatory region comprises a contiguous span of at least 30 nucleotides of SEQ ID No 1 or the complements thereof, or homologs thereof having at least 80% nucleotide identity.

15. The method of claim 9, wherein said p110β regulatory region comprises a contiguous span of at least 30 nucleotides of SEQ ID No 1 or the complements thereof, or homologs thereof having at least 90% nucleotide identity.

16. The method of claim 9, wherein said p110β regulatory region comprises a contiguous span of at least 30 nucleotides of SEQ ID No 1 or the complements thereof, or homologs thereof having at least 95% nucleotide identity.

17. The method of any of claim 1, wherein the subject is an obese subject.

18. The method of any of claim 1, wherein the subject is a human subject.

19. The method of claim 18, wherein the subject has a BMI of at least 25.

20. The method of claim 18, wherein the subject has a BMI of at least 30.

21. The method of claim 18, wherein the subject has a BMI of at least 35.

22. The method of claim 2, wherein said agent for the prevention or treatment of insulin resistance is a compound of the thiazolidinedione class.

23. The method of claim 9, wherein said test compound is a compound of the thiazolidinedione class.

24. The method of claim 3, wherein an increased level of p110β expression is correlated with a decreased likelihood of developing insulin resistance, thereby identifying the subject as having a decreased likelihood of developing insulin resistance.

25. The method of claim 1, wherein the subject is a child.

26. The method of claim 3, wherein the subject is a child.

27. The method of claim 5, wherein the subject is a child.
